(19) United States
(12) Patent Application Publication
(10) Pub. No.: US 2024/0238290 A1
(43) Pub. Date: Jul. 18, 2024

(54) USE OF COMPLEMENT FACTOR D INHIBITOR FOR TREATMENT OF GENERALIZED MYASTHENIA GRAVIS

(71) Applicant: Alexion Pharmaceuticals, Inc., Boston, MA (US)

(72) Inventors: Mingjun Huang, New Haven, CT (US); Laura Marie Gault, Hingham, MA (US); Marcus Ryan Yountz, Needham, MA (US); Moshe Vardi, Newton, MA (US); Cynthia Carrillo-Infante, Winchester, MA (US)

(73) Assignee: Alexion Pharmaceuticals, Inc., Boston, MA (US)

(21) Appl. No.: 18/559,420

(22) PCT Filed: May 3, 2022
(86) PCT No.: PCT/US2022/027408

§ 371 Date: Nov. 7, 2023

Related U.S. Application Data
(60) Provisional application No. 63/186,301, filed on May 10, 2021.

(51) Int. Cl. A61K 31/506; A61P 21/04

(52) U.S. Cl. CPC A61K 31/506; A61P 21/04

(57) ABSTRACT

Disclosed herein are methods for treating myasthenia gravis (MG) in a subject. The methods include administering to the subject a therapeutically effective amount of a small molecule complement factor D inhibitor.

### CROSS-REFERENCE TO RELATED APPLICATIONS

This application claims priority to, and the benefit of, U.S. Provisional Application No. 63/186,301, filed May 10, 2021, the entire contents which are incorporated herein by reference.

### BACKGROUND

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions.

Myasthenia Gravis (MG) is a rare, debilitating, acquired autoimmune neurologic disorder of the neuromuscular junction (NMJ) caused by the failure of neuromuscular transmission, which results from the binding of auto-antibodies (auto-Abs) to proteins involved in signaling at the NMJ. These proteins include the nicotine acetylcholine receptors (AChRs) or, less frequently, a muscle-specific tyrosine kinase (MuSK) involved AChR clustering.

MG may cause life-threatening respiratory failure, referred to as myasthenic crisis. MG has a prevalence of 14-20 per 100,000 in the U.S., affecting roughly 60,000 Americans. It affects males and females in equal ratio, although the incidence in females peaks in the 3^{rd} decade as compared to males in whom the peak age at onset is in the 6th or 7th decade. About 15% to 20% of subjects will experience a myasthenic crisis during the course of their disease, 75% within 2 years of diagnosis, requiring hospitalization and ventilator support. Mortality from MG is approximately 4%, mostly due to respiratory failure.

Myasthenia gravis is clinically characterized by weakness and fatigability of voluntary skeletal muscles. MG may initially present with ocular muscle weakness affecting eye and eyelid movement, referred to as ocular MG (oMG). Ten percent of subjects have disease limited to ocular muscles. Ninety percent of subjects have generalized MG, with muscle weakness involving neck, head, spine, bulbar, respiratory or limb muscles. Bulbar weakness refers to muscles controlled by nerves originating from the bulb-like part of the brainstem and manifests as difficulty in talking, chewing, swallowing and control of the head.

Generalized myasthenia gravis (gMG) patients differ from the ocular myasthenia gravis (oMG) population in that neuromuscular inflammation and the resultant clinical findings are not just limited to the ocular muscles, but involve all voluntary muscle groups: the bulbar, respiratory, head, neck, trunk or peripheral muscles with or without involvement of the eyes. Profound weakness and devastating consequences, including slurred speech, dysarthria, dysphagia, disorienting vision, shortness of breath (both with activity and at rest), weakness of the upper and lower extremities, impaired mobility, marked reductions in the ability to perform activities of daily living (ADLs), extreme fatigue and episodes of pulmonary failure requiring mechanical ventilation are hallmarks of gMG. Compared with patients with isolated oMG, patients with gMG have a greater incidence of morbidities and a higher burden of disease. gMG is a rare disorder, having an estimated prevalence between 145 to 278 per million. Patients with gMG suffer from a devastating inflammatory neuromuscular disorder with limited therapeutic options.

Hospitalizations for gMG exacerbations are common, with the need for respiratory support, including mechanical ventilation secondary to respiratory failure (e.g., myasthenic crisis) and gastrointestinal tube placement for nutritional support and prevention of dysphagia-associated aspiration. Patients with more advanced gMG have been reported to experience increased mortality of up to 40% at 10 years following diagnosis.

While there is no cure for MG, there are therapies that reduce muscle weakness and improve neuromuscular function. Currently available treatments for myasthenia gravis aim to modulate neuromuscular transmission, inhibit the production or effects of pathogenic antibodies, or inhibit inflammatory cytokines. There is currently no specific treatment that targets the underlying pathophysiology of NMJ injury, specifically anti-AChR antibody-AChR interactions resulting in complement activation via the classical pathway and inflammation, with the resultant destruction of the NMJ. There is no specific treatment that corrects the autoimmune defect in MG. With immunosuppressive therapies (ISTs) representing the current standard of care, which usually combines cholinesterase inhibitors, corticosteroids and immunosuppressive drugs (most commonly azathioprine (AZA), cyclosporine, and mycophenolate mofetil (MMF)), the majority of subjects with MG can have their disease reasonably controlled. These therapies, however, may not be optimal for all patients, and there is a cohort of subjects who do not respond adequately to ISTs, or cannot tolerate ISTs, and those who require repeated treatments with plasma exchange (PE) and/or intravenous immunoglobulin (IVIg) to maintain clinical stability.

In difficult-to-control cases, patients with gMG experience unrelenting inflammation, tissue destruction, and consequent severe morbidities including profound muscle weakness, impaired mobility, shortness of breath, pulmonary failure, extreme fatigue, risk for aspiration, and markedly impaired ADLs. These patients are typically diagnosed in the prime of their adult lives, with a median age of onset ranging from 36 to 60 years. As a result of the morbidities associated with gMG, many patients cannot work or have diminished work capacity, experience difficultly caring for themselves and others, and require assistance speaking, eating, ambulating, breathing and performing ADLs.

Uncontrolled terminal complement activation has been implicated in animal models of experimental autoimmune gMG as well as in other forms of autoimmune neuropathy in humans. Auto-Abs recognize targeted neural or muscle tissues, including the AChR, leading to uncontrolled terminal complement activation at the neural or muscle surface. Autoantibody-driven uncontrolled terminal complement activation with membrane attack complex (MAC)-dependent lysis and activation, and C5a-dependent inflammation at the NMJ causes AChR loss and failure of neuromuscular transmission. Consistent with this model, both complement component C3 fragments (C3a and C3b) and the MAC C5b-9 have been found in NMJs of MG patients.

As there is no cure for MG, and standard of care is not effective for all patients, there is a need to provide improved methods for treating these patients.

### SUMMARY

Provided herein are methods for treating myasthenia gravis (MG), e.g., generalized myasthenia gravis (gMG), in a subject with a small molecule complement factor D (CFD) inhibitor. The present disclosure is based, in part, on the use of oral CFD inhibitors, such as Compound 1 or a pharmaceutically acceptable salt thereof, as a first-in-class treatment option for patients with MG. The present inventors have identified that although FD is the least abundant complement protein, it nonetheless serves as an ideal pharmaceutical target in the therapy of MG because it plays an important role as the rate limiting component of the AP. Compound 1 is a potent inhibitor of FD with demonstrated dose dependent inhibition of complement AP activity in vivo. Biomarkers of AP activity, e.g., plasma Bb levels, show significant dose-dependent reductions following administration of Compound 1, thus making it a useful therapeutic in the treatment of MG. As an orally administered small molecule, Compound 1 is advantageous over many approved medications as well as those in development, which rely on intravenous (IV) or subcutaneous (SC) administration. In this regard, oral CFD inhibitors, such as Compound 1, not only provide MG patients with a more convenient and accessible option for therapy, but also help reduce the treatment burden for patients and their families. Given the overall pharmaceutical profile of Compound 1, the present inventors contemplate that a treatment regimen with Compound 1 will lead to better patient compliance and clinical outcomes especially in patients with severe disease, e.g., MG characterized by impaired mobility, vision, and/or dexterity.

In a first aspect, the disclosure features a method of treating MG in a subject, which includes administering to the subject a therapeutically effective amount of Compound 1:

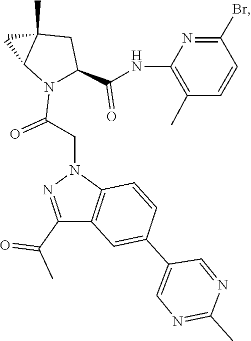 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]

or a pharmaceutically acceptable salt thereof.

In some embodiments, the subject was diagnosed with MG at least 3 months prior to receiving treatment. The MG diagnosis may be confirmed via (i) a positive serologic test for anti-AChR antibodies and an abnormal neuromuscular transmission demonstrated by single fiber electromyography or repetitive nerve stimulation; (ii) a positive response to an acetylcholinesterase inhibitor (AChEI) test; or (ii) an improvement of signs or symptoms related to MG during treatment with an oral AChEI, as determined by a treating physician.

In some embodiments, the subject is classified as having Myasthenia Gravis Foundation of America (MGFA) class II to IV disease.

In some embodiments, the subject has a MG activities of daily living (MG-ADL) score of ≥5.

In some embodiments, the subject has been receiving treatment with (i) azathioprine for ≥6 months, with a stable dose of ≥2 months; (ii) an immunosuppressant (e.g., mycophenolate mofetil, methotrexate, or cyclophosphamide) for ≥3 months, with a stable does for ≥1 month; (iii) a corticosteroid (e.g., prednisone at a maximum dose of 20 mg/day or an equivalent thereof) with a stable dose for ≥4 weeks; or (iv) an AChEI with a stable dose for ≥2 weeks.

In some embodiments, Compound 1 or the pharmaceutically acceptable salt thereof is orally administered, e.g., at a dose of about 60 mg to about 300 mg BID (e.g., about 80 mg to about 250 mg BID, about 100 mg to about 200 mg BID, about 120 mg to about 180 mg BID, about 60 mg BID, about 70 mg BID, about 80 mg BID, about 90 mg BID, about 100 mg BID, about 110 mg BID, about 120 mg BID, about 130 mg BID, about 140 mg BID, about 150 mg BID, about 160 mg BID, about 170 mg BID, about 180 mg BID, about 190 mg BID, about 200 mg BID, about 210 mg BID, about 220 mg BID, about 230 mg BID, about 240 mg BID, about 250 mg BID, about 260 mg BID, about 270 mg BID, about 280 mg BID, about 290 mg BID, or about 300 mg BID.

In some embodiments, the treatment results in the subject experiencing a clinically meaningful improvement as determined by a reduction in Myasthenia Gravis Activities of Daily Living (MG-ADL) score. In some embodiments, the MG-ADL score is reduced by at least 2 points (e.g., at least 3, 4, 5, 6, 7, or 8 points) in 4 consecutive weeks after 8 weeks of treatment, and the subject has not received a rescue therapy (i.e., high-dose corticosteroid, plasma exchange (PE)/plasmapheresis (PP), or intravenous immunoglobulin (IVIg) therapy) during treatment. In some embodiments, the MG-ADL score is reduced by at least 2 points (e.g., 2, 3, 4, 5, 6, 7, or 8 points) after 8 weeks of treatment. In some embodiments, the MG-ADL score is reduced after 26 weeks of treatment by, e.g., at least 2 points (e.g., at least 3, 4, 5, 6, 7, or 8 points).

In some embodiments, the treatment results in the subject experiencing a clinically meaningful improvement as determined by a reduction in quantitative Myasthenia Gravis (QMG) score after 8 weeks of treatment. In some embodiments, the QMG score is reduced by at least 3 points (e.g. at least 4, 5, 6, 7, or 8 points) after 8 weeks of treatment. In some embodiments, the QMG score is reduced by at least 3 points (e.g., at least 4, 5, 6, 7, or 8 points) in four consecutive weeks after 8 weeks of treatment, and the subject has not received a rescue therapy (i.e., high-dose corticosteroid, PE/PP, or IVIg therapy) during treatment.

In some embodiments, the treatment results in the subject experiencing a clinically meaningful improvement as determined by (a) a reduction in MG-ADL score of at least 2 points in 4 consecutive weeks after 8 weeks of treatment without a need for a rescue therapy during treatment and (b) a reduction in QMG score characterized by (1) change from baseline in QMG total score after 8 weeks of treatment; (2) at least a 3 point improvement in the QMG total score at week 8; and/or (3) at least a 3 point improvement in the QMG total score in any 4 consecutive weeks during the first 8 weeks without a need for rescue therapy,

In some embodiments, the treatment results in the subject experiencing a clinically meaningful improvement as determined by a reduction in Quality of Life in Neurological Disorders (Neuro-QoL) Fatigue score after 8 weeks of treatment.

In some embodiments, the treatment results in the subject experiencing a clinically meaningful improvement as determined by (a) a reduction in MG-ADL score of at least 2 points in 4 consecutive weeks after 8 weeks of treatment without a need for a rescue therapy during treatment; (b) a reduction in QMG score characterized by (1) change from baseline in QMG total score after 8 weeks of treatment; (2) at least a 3 point improvement in the QMG total score at week 8; and/or (3) at least a 3 point improvement in the QMG total score in any 4 consecutive weeks during the first 8 weeks without a need for rescue therapy; and (c) a reduction in Neuro-QoL Fatigue score after 8 weeks of treatment,

In some embodiments, the treatment results in the subject experiencing a clinically meaningful improvement as determined by an improvement in the Myasthenia Gravis Foundation of America Post-Intervention Status (MGFA-PIS).

In some embodiments, the treatment results in the subject experiencing a clinically meaningful improvement as determined by (a) a reduction in MG-ADL score of at least 2 points in 4 consecutive weeks after 8 weeks of treatment without a need for a rescue therapy during treatment; (b) a reduction in QMG score characterized by (1) change from baseline in QMG total score after 8 weeks of treatment; (2) at least a 3 point improvement in the QMG total score at week 8; and/or (3) at least a 3 point improvement in the QMG total score in any 4 consecutive weeks during the first 8 weeks without a need for rescue therapy; (c) a reduction in Neuro-QoL Fatigue score after 8 weeks of treatment; and (d) an improvement in the MGFA-PIS.

In some embodiments, the MG is generalized myasthenia gravis (gMG).

In some embodiments, the subject anti-AChR antibody positive.

In some embodiments, the subject has a history of thymectomy, thymomectomy, or any other thymic surgery within 12 months prior to the treatment of MG.

In some embodiments, the subject has an untreated thymic malignancy, carcinoma, or thymoma.

In some embodiments, the subject has a history of treatment thymic malignancy or carcinoma, and (a) treatment of the thymic malignancy or carcinoma was completed more than 5 years prior to the treatment of MG; (b) there is no known recurrence of the thymic malignancy or carcinoma within 5 years prior to the treatment of MG; and (c) there is no radiological indication of recurrence of the thymic malignancy or carcinoma in a computed tomography (CT) or magnetic resonance imaging (MRI) scan performed within 6 months prior to the treatment of MG.

In some embodiments, the subject has a history of treated benign thymoma, and (a) the subject has histopathological or equivalent records indication the diagnosis of benign thymoma; (b) treatment of the benign thymoma was completed more than 12 months prior to the treatment of MG; (c) there is no known recurrence of the benign thymoma within 12 years prior to the treatment of MG; and (d) there is no radiological indication of recurrence of the benign thymoma in a CT or MRI scan performed within 6 months prior to the treatment of MG.

In some embodiments, the subject does not have a history of malignancy within 5 years prior to treatment, wherein the malignancy is not nonmelanoma skin cancer or carcinoma in situ of the cervix that has been treated with no evidence of recurrence.

In some embodiments, the subject is not exhibiting clinical features consistent with clinical deterioration prior to the treatment of MG.

In some embodiments, the subject does not have a history of seizure.

In some embodiments, the subject does not have a history of N meningitidis infection.

In some embodiments, the subject is not exhibiting signs of a human immunodeficiency virus infection (e.g., HIV antibody positive).

In some embodiments, the subject is not exhibiting signs of a hepatitis B viral infection (positive hepatitis surface antigen or positive core antibody) with negative surface antibodies.

In some embodiments, the subject is not exhibiting signs of a hepatitis C viral infection (HCV antibody positive); or is exhibiting signs of a hepatitis C viral infection but has been successfully treated and has a documented sustained virologic response.

In some embodiments, the subject does not have a history of persistent or recurrent infections.

In some embodiments, the subject did not have an active systemic bacterial, viral, or fungal infection within 14 days prior to treatment.

In some embodiments, the subject does not have a history of or have risk factors for Torsades de Pointes (e.g., heart failure/cardiomyopathy or family history of Long QT Syndrome), a QT interval corrected using Fridericia's formula (QTcF) >450 msec when the subject is male or >470 msec when the subject is female, or is receiving medication known to significantly increase the corrected QT interval (QTc).

In some embodiments, the subject does not have an alanine aminotransferase level of >2×upper limit of normal (ULN).

In some embodiments, the subject does not have a direct bilirubin level of >2×ULN.

In some embodiments, the subject has not received intravenous immunoglobulin (IVIg) or subcutaneous immunoglobulin (SCIg) therapy within 4 weeks prior to the treatment with MG.

In some embodiments, the subject has not received plasma exchange/plasmapheresis (PE/PP) treatment within 4 weeks prior to the treatment with MG.

In some embodiments, the subject has not received treatment with rituximab within 6 months prior to the treatment with MG.

In some embodiments, the subject has not received treatment tacrolimus or cyclosporine within 4 weeks prior to the treatment with MG.

In some embodiments, the subject has not received or is not receiving treatment with a complement inhibitor.

In some embodiments, the subject has not received treatment with a medication selected from a strong cytochrome P450, family 3, subfamily A (CYP3A) inhibitor; a moderate CYP3A inhibitor; a strong inducer of CYP3A; a moderate inducer of CYP3A; and a sensitive substrate of CYP3A, within the longer of two weeks or five half-lives of the medication prior to the treatment of MG. Exemplary medications are provided in Table 13.

In some embodiments, the subject has not received treatment with a medication selected from meperidine, pethidine, a typical (1^{st} generation) antipsychotic, clozapine, olanzapine, lithium, a tricyclic antidepressant, bupropion, aminophylline, and theophylline.

In some embodiments, the subject is not receiving treatment with a biologic medication that may affect immune system function; or the subject was previously receiving treatment with a biologic medication that may affect immune system function, and the treatment has ended for at least 5 terminal half-lives of the biologic medication.

In some embodiments, the subject is restricted from consuming foods and beverages (e.g., grapefruit) that inhibit CYP3A4 enzyme activity.

In some embodiments, the subject is restricted from using a medication selected from a strong CYP3A inhibitor, a moderate CYP3A inhibitor, a strong inducer of CYP3A, a moderate inducer of CYP3A, and a sensitive substrate of CYP3A. Exemplary medications are provided in Table 13.

In some embodiments, the subject is restricted from using a medication selected from meperidine, pethidine, a typical (1^{st} generation) antipsychotic, clozapine, olanzapine, lithium, a tricyclic antidepressant, bupropion, aminophylline, and theophylline.

In some embodiments, the subject is restricted from using a medication selected from a strong CYP3A inhibitor, a moderate CYP3A inhibitor; a strong inducer of CYP3A; a moderate inducer of CYP3A; and a sensitive substrate of CYP3A.

In some embodiments, the subject is restricted from using a medication selected from meperidine, pethidine, a typical (1^{st} generation) antipsychotic, clozapine, olanzapine, lithium, a tricyclic antidepressant, bupropion, aminophylline, and theophylline.

In some embodiments, the subject is restricted from using IVIg or SCIg as a maintenance therapy.

In some embodiments, the subject is restricted from using PE/PP as a maintenance therapy.

In some embodiments, the subject is restricted from receiving treatment with rituximab.

In some embodiments, the subject is restricted from receiving treatment with tacrolimus or cyclosporine.

In some embodiments, the subject is restricted from receiving treatment with a complement inhibitor other than Compound 1 or the pharmaceutically acceptable thereof.

In some embodiments, the subject is restricted from receiving treatment with a biologic medication that my affect immune system function.

In some embodiments, the subject has vaccinated against meningococcal infections (a) within 3 years and more than two weeks prior to the treating of MG; or (b) less than two weeks prior to the treatment of MG, and the subject is treated with an appropriate prophylactic antibiotic until at least 2 weeks after vaccination.

In a second aspect, the disclosure features the use of Compound 1 or a pharmaceutically acceptable salt thereof in the manufacture of a medicament for use in a method of treating MG in a subject (e.g., any one of the methods disclosed herein).

In a third aspect, the disclosure features a compound for use in a method of treating MG in a subject (e.g., any one of the methods disclosed herein), wherein the compound is Compound 1 or a pharmaceutically acceptable salt thereof.

In a fourth aspect, the disclosure features a kit for treating MG in a subject comprising (a) a dose of Compound 1 or a pharmaceutically acceptable salt thereof, and (b) instructions for using Compound 1 or the pharmaceutically acceptable salt thereof according to any one or more of the methods disclosed herein.

The following non-limiting embodiments are specifically provided herein: In some embodiments, the disclosure relates to a method of treating myasthenia gravis (MG), particularly generalized myasthenia gravis (gMG), in a subject, comprising administering to the subject a therapeutically effective amount of Compound 1 or a pharmaceutically acceptable salt thereof, wherein Compound 1 or the pharmaceutically acceptable salt thereof is administered at a dose of about 60 mg to about 300 mg BID; particularly wherein Compound 1 or the pharmaceutically acceptable salt thereof is orally administered.

In some embodiments according to the foregoing or the following, the administering of Compound 1 or the pharmaceutically acceptable salt thereof results in the treatment of subject's MG; particularly treatment of the subject's gMG.

In some embodiments according to the foregoing or the following, the subject was diagnosed with MG at least 3 months prior to receiving treatment.

In some embodiments according to the foregoing or the following, the MG diagnosis is confirmed via a positive serologic test for anti-AChR antibodies and an abnormal neuromuscular transmission demonstrated by single fiber electromyography or repetitive nerve stimulation; via a positive response to an acetylcholinesterase inhibitor (AChEI) test; and/or via an improvement of signs or symptoms related to MG during treatment with an oral AChEI, as determined by a treating physician.

In some embodiments according to the foregoing or the following, the subject is classified as having Myasthenia Gravis Foundation of America (MGFA) class II to IV disease.

In some embodiments according to the foregoing or the following, the subject has a MG activities of daily living (MG-ADL) score of ≥5.

In some embodiments according to the foregoing or the following, the subject has been receiving treatment with azathioprine for ≥6 months, with a stable dose of ≥2 months.

In some embodiments according to the foregoing or the following, the subject has been receiving treatment with an immunosuppressant for ≥3 months, with a stable does for ≥1 month, particularly wherein the immunosuppressant is mycophenolate mofetil or methotrexate or cyclophosphamide.

In some embodiments according to the foregoing or the following, the subject has been receiving treatment with a corticosteroid with a stable dose for ≥4 weeks, particularly wherein the corticosteroid is prednisone at a maximum dose of 20 mg/day or an equivalent thereof.

In some embodiments according to the foregoing or the following, the subject has been receiving treatment with an AChEI with a stable dose for ≥2 weeks.

In some embodiments according to the foregoing or the following, the Compound 1 or the pharmaceutically acceptable salt thereof is administered at a dose of about 80 mg to about 250 mg BID; particularly at a dose of about 120 mg BID; more particularly at a dose of about 180 mg BID.

In some embodiments according to the foregoing or the following, the treatment results in the subject experiencing a clinically meaningful improvement as determined by a reduction in Myasthenia Gravis Activities of Daily Living (MG-ADL) score, e.g., by at least 2 points in 4 consecutive weeks after 8 weeks of treatment, and the subject has not received a rescue therapy during treatment or by at least 2 points after 8 weeks of treatment; or by at least 2 points after 26 weeks of treatment.

In some embodiments according to the foregoing or the following, the treatment results in the subject experiencing a clinically meaningful improvement as determined by a reduction in quantitative Myasthenia Gravis (QMG) score after 8 weeks of treatment, e.g., by at least 3 points after 8 weeks of treatment; by at least 3 points in four consecutive weeks after 8 weeks of treatment, and the subject has not received a rescue therapy during treatment.

In some embodiments according to the foregoing or the following, the treatment results in the subject experiencing a clinically meaningful improvement as determined by a reduction in Quality of Life in Neurological Disorders (Neuro-QoL™) Fatigue score after 8 weeks of treatment.

In some embodiments according to the foregoing or the following, the treatment results in the subject experiencing a clinically meaningful improvement as determined by an improvement in the Myasthenia Gravis Foundation of America Post-Intervention Status (MGFA-PIS).

In some embodiments according to the foregoing or the following, the subject is anti-AChR antibody positive; has a history of thymectomy, thymomectomy, or any other thymic surgery within 12 months prior to the treatment of MG; has an untreated thymic malignancy, carcinoma, or thymoma; or has a history of treatment thymic malignancy or carcinoma, wherein: (a) treatment of the thymic malignancy or carcinoma was completed more than 5 years prior to the treatment of MG; (b) there is no known recurrence of the thymic malignancy or carcinoma within 5 years prior to the treatment of MG; and/or (c) there is no radiological indication of recurrence of the thymic malignancy or carcinoma in a computed tomography (CT) or magnetic resonance imaging (MRI) scan performed within 6 months prior to the treatment of MG.

In some embodiments according to the foregoing or the following, the subject has a history of treated benign thymoma, wherein: (a) the subject has histopathological or equivalent records indication the diagnosis of benign thymoma; (b) treatment of the benign thymoma was completed more than 12 months prior to the treatment of MG; (c) there is no known recurrence of the benign thymoma within 12 years prior to the treatment of MG; and/or (d) there is no radiological indication of recurrence of the benign thymoma in a CT or MRI scan performed within 6 months prior to the treatment of MG.

In some embodiments according to the foregoing or the following, the subject does not have a history of malignancy within 5 years prior to treatment, wherein the malignancy is not nonmelanoma skin cancer or carcinoma in situ of the cervix that has been treated with no evidence of recurrence.

In some embodiments according to the foregoing or the following, the subject is not exhibiting clinical features consistent with clinical deterioration prior to the treatment of MG, e.g., does not have a history of seizure. In some embodiments according to the foregoing or the following, the subject does not have a history of N meningitidis infection; human immunodeficiency virus infection; hepatitis B viral infection with negative surface antibodies; hepatitis C viral infection; or is exhibiting signs of a hepatitis C viral infection but has been successfully treated and has a documented sustained virologic response; a history of persistent or recurrent infections; an active systemic bacterial, viral, or fungal infection within 14 days prior to treatment; a history of or have risk factors for Torsades de Pointes, a QT interval corrected using Fridericia's formula (QTcF) >450 msec when the subject is male or >470 msec when the subject is female, or is receiving medication known to significantly increase the corrected QT interval (QTc); alanine aminotransferase level of >2×ULN; direct bilirubin level of >2×ULN; has not received intravenous immunoglobulin (IVIg) or subcutaneous immunoglobulin (SCIg) therapy within 4 weeks prior to the treatment with MG; has not received plasma exchange/plasmapheresis (PE/PP) treatment within 4 weeks prior to the treatment with MG; has not received treatment with rituximab within 6 months prior to the treatment with MG; has not received treatment tacrolimus or cyclosporine within 4 weeks prior to the treatment with MG; has not received or is not receiving treatment with a complement inhibitor; has not received treatment with a medication selected from a strong cytochrome P450, family 3, subfamily A (CYP3A) inhibitor; a moderate CYP3A inhibitor; a strong inducer of CYP3A; a moderate inducer of CYP3A; and a sensitive substrate of CYP3A, within the longer of two weeks or five half-lives of the medication prior to the treatment of MG; has not received treatment with a medication selected from meperidine, pethidine, a typical (1st generation) antipsychotic, clozapine, olanzapine, lithium, a tricyclic antidepressant, bupropion, aminophylline, and theophylline; and/or is not receiving treatment with a biologic medication that may affect immune system function; or the subject was previously receiving treatment with a biologic medication that may affect immune system function, and the treatment has ended for at least 5 terminal half-lives of the biologic medication.

In some embodiments according to the foregoing or the following, the subject is restricted from: consuming foods and beverages that inhibit CYP3A4 enzyme activity; using a medication selected from a strong CYP3A inhibitor, a moderate CYP3A inhibitor, a strong inducer of CYP3A, a moderate inducer of CYP3A, and a sensitive substrate of CYP3A; using a medication selected from meperidine, pethidine, a typical (1st generation) antipsychotic, clozapine, olanzapine, lithium, a tricyclic antidepressant, bupropion, aminophylline, and theophylline; using a medication selected from a strong CYP3A inhibitor, a moderate CYP3A inhibitor; a strong inducer of CYP3A; a moderate inducer of CYP3A; and/or a sensitive substrate of CYP3A; using a medication selected from meperidine, pethidine, a typical (1st generation) antipsychotic, clozapine, olanzapine, lithium, a tricyclic antidepressant, bupropion, aminophylline, and theophylline; using IVIg or SCIg as a maintenance therapy; using PE/PP as a maintenance therapy; receiving treatment with rituximab; receiving treatment with tacrolimus or cyclosporine; receiving treatment with a complement inhibitor other than Compound 1 or the pharmaceutically acceptable thereof; receiving treatment with a biologic medication that my affect immune system function.

In some embodiments according to the foregoing or the following, the subject has vaccinated against meningococcal infections: (a) within 3 years and more than two weeks prior to the treating of MG; or (b) less than two weeks prior to the treatment of MG, and the subject is treated with an appropriate prophylactic antibiotic until at least 2 weeks after vaccination.

In some embodiments, the disclosure relates to the use of Compound 1 or a pharmaceutically acceptable salt thereof in the manufacture of a medicament for use in a method of treating MG in a subject (e.g., in accordance with any of the foregoing methods).

In some embodiments, the disclosure relates to Compound 1 or a pharmaceutically acceptable salt thereof for use in a method of treating MG in a subject (e.g., in accordance with any of the foregoing methods).

### BRIEF DESCRIPTIONS OF THE DRAWING

FIG. 1 is a schematic depicting the design of a Phase II clinical trial described in Example 1 (EOS: end of study).

### DETAILED DESCRIPTION

### Definitions

As used herein, the word “a” or “plurality” before a noun represents one or more of the particular nouns. For example, the phrase “a mammalian cell” represents “one or more mammalian cells.” The singular form “a,” “an,” and “the” include plural reference unless the context clearly dictates otherwise.

The term “about”, particularly in reference to a given quantity or number, is meant to encompass deviations within plus or minus ten percent (±10%), (e.g., ±5%).

As used herein, the phrase “clinical deterioration” refers to patients who experience an MG crisis, which is defined as weakness from MG that is severe enough to necessitate intubation or to delay extubation following surgery, where the respiratory failure is due to weakness of respiratory muscles, severe bulbar (oropharyngeal) muscle weakness accompanies the respiratory muscle weakness, or is the predominant feature in a patient; or when there is significant symptomatic worsening to a score of 3 or a 2-point worsening from baseline on any one of the individual MG-Activities of Daily Living (MG-ADL) items other than double vision or eyelid droop; or administration of rescue therapy is provided to a patient whose health, in the opinion of the investigator or investigator-designated physician, would be in jeopardy, if rescue therapy were not given (e.g., emergent situations).

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and CG. Wermuth), Wiley-VCH, 2008. These salts may be acid addition salts involving inorganic or organic acids. The salts can be prepared in situ during the final isolation and purification of the compounds described herein or separately by reacting the free base group with a suitable acid. Methods for preparation of the appropriate salts are well-established in the art. Representative acid addition salts include acetate, adipate, alginate, ascorbate, aspartate, benzenesulfonate, benzoate, bisulfate, borate, bromide, butyrate, camphorate, camphorsulfonate, chloride, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like.

As used herein, the term “pharmaceutical composition” refers to an active compound, formulated together with one or more pharmaceutically acceptable excipients. In some embodiments, a compound is present in unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained release formulation; topical application, for example, as a cream, ointment, or a controlled release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The term “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Excipients include, but are not limited to: butylated optionally substituted hydroxytoluene (e.g., BHT), calcium carbonate, calcium phosphate dibasic, calcium stearate, croscarmellose, crosslinked polyvinyl pyrrolidone, citric acid, crospovidone, cysteine, ethylcellulose, gelatin, optionally substituted hydroxypropyl cellulose, optionally substituted hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch, stearic acid, stearic acid, sucrose, talc, titanium dioxide, vitamin A, vitamin E, vitamin C, and xylitol. Those of ordinary skill in the art are familiar with a variety of agents and materials useful as excipients.

As used herein, the phrase “requires chronic plasma exchange” to maintain clinical stability refers to the use of plasma exchange therapy on a patient on a regular basis for the management of muscle weakness at least every 3 months over the last 1 months.

As used herein, the phrase “requires chronic IVIg” to maintain clinical stability refers to the use of IVIg therapy on a patient on a regular basis for the management of muscle weakness at least every 3 months over the last 1 months. In certain embodiments, treatment of MG includes the amelioration or improvement of one or more symptoms associated with MG. Symptoms associated with MG include muscle weakness and fatigability. Muscles primarily affected by MG include muscles that control eye and eyelid movement, facial expressions, chewing, talking, swallowing, breathing, neck movements, and limb movements.

As used herein, the term “subject” or “patient” is a human patient (e.g., a patient having myasthenia gravis (MG)). As used herein, the terms “subject” and “patient” are interchangeable.

As used herein, the term “treating” includes therapeutic treatments. The term “therapeutic” treatment is art-recognized and includes administration to a human subject of one or more of the disclosed compounds or formulations after manifestation of the unwanted condition (i.e., it is intended to diminish, ameliorate, or stabilize the existing unwanted condition or side effects thereof). Preferably, it is intended that the severity of the subject's condition (e.g., diminished ability to speak or swallow; weakness of neck and limb muscles; breathing difficulty; and/or increased general fatigue) is reduced or at least partially improved or modified and that some alleviation, mitigation, reversal or decrease in at least one clinical symptom (e.g., general fatigue) is achieved.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. Effective treatment may refer to, for example, alleviation of at least one symptom of MG.

The term “effective amount” or “therapeutically effective amount” refers to an amount of an agent that provides the desired biological, therapeutic, and/or prophylactic result. That result can be reduction, amelioration, palliation, lessening, delaying and/or alleviation of one or more of the signs, symptoms, or causes of a disease, or any other desired alteration of a biological system. In one example, an “effective amount” is the amount of Compound 1 or a pharmaceutically acceptable salt thereof useful, e.g., clinically proven, to alleviate at least one symptom of MG. An effective amount can be administered in one or more administrations.

### Method of Treating Myasthenia Gravis

The disclosure provides methods for treating subjects suffering from myasthenia gravis (MG) by administering to the subject a therapeutically effective amount of Compound 1, or a pharmaceutically acceptable salt thereof.

In some embodiments, the MG is gMG. In some embodiments, the gMG is refractory gMG. In some embodiments, refractory gMG is characterized as including subjects or patients positive for auto-antibodies binding to nicotinic acetylcholine receptor (anti-AChR antibodies) who continue to show marked generalized weakness or bulbar signs and symptoms of MG while receiving current standard of care for myasthenia gravis such as cholinesterase inhibitor therapy and immunosuppressant therapy (IST) or who require chronic plasma exchange or chronic IVIg to maintain clinical stability. In other embodiments, refractory gMG is characterized as including subjects or patients who continue to show marked generalized weakness or bulbar signs and symptoms of MG while receiving current standard of care for MG such as cholinesterase inhibitor therapy and immunosuppressant therapy (IST) or who require chronic plasma exchange or chronic IVIg to maintain clinical stability.

In some embodiments, treatment of MG includes the improvement of a clinical marker for MG progression. These markers include MG activity of daily living profile (MG-ADL), quantitative Myasthenia Gravis (QMG) score for Disease Severity, negative inspiratory force (NIF), forced vital capacity (FVC), MGFA postintervention status (MGFA-PIS), and other quality of life measurements. In some embodiments, MG-ADL is the primary score for measuring improvement of MG.

The MG-ADL is an 8-point questionnaire that focuses on relevant symptoms and functional performance of activities of daily living (ADL) in MG subjects (Table 1). The 8 items of the MG-ADL were derived from symptom-based components of the original 13-item QMG to assess disability secondary to ocular (2 items), bulbar (3 items), respiratory (1 item), and gross motor or limb (2 items) impairment related to effects from MG. In this functional status instrument, each response is graded 0 (normal) to 3 (most severe). The range of total MG-ADL score is 0-24. A clinically meaningful improvement in a patient's MGADL would be a 3 point or greater reduction in score after 26 weeks of treatment.

TABLE 1
MG Activity of Daily Living (MG-ADL) Profile

| Items | Grade 0 | Grade 1 | Grade 2 | Grade 3 |
|---|---|---|---|---|
| 1. Talking | Normal | Intermittent slurring | Constant slurring or | Difficult to |
| | | or nasal speech | nasal speech, but | understand speech |
| | | | can be understood | |
| 2. Chewing | Normal | Fatigue with solid | Fatigue with soft | Gastric tube |
| | | food | food | |
| 3. Swallowing | Normal | Rare episode of | Frequent choking | Gastric tube |
| | | choking | necessitating | |
| | | | changes in diet | |
| 4. Breathing | Normal | Shortness of breath | Shortness of breath | Ventilator |
| | | with exertion | at rest | dependence |
| 5. Impairment of | None | Extra effort, but no | Rest periods | Cannot do one of |
| ability to brush teeth | | rest periods needed | needed | these functions |
| or comb hair | | | | |
| 6. Impairment of | None | Mild, sometimes | Moderate, always | Severe, requires |
| ability to arise from | | uses arms | uses arms | assistance |
| a chair | | | | |
| 7. Double vision | None | Occurs, but not daily | Daily, but not | Constant |
| | | | constant | |
| 8. Eyelid drop | None | Occurs, but not daily | Daily, but no | Constant |
| | | | constant | |

The QMG Score for Disease Severity is a scoring system consists of 13 items: ocular (2 items), facial (1 item), bulbar (2 items), gross motor (6 items), axial (1 item), and respiratory (1 item); each graded 0 to 3, with 3 being the most severe (Table 2). The range of total QMG score is 0-39.

The QMG scoring system is considered to be an objective evaluation of therapy for MG and is based on quantitative testing of sentinel muscle groups. The MGFA task force has recommended that the QMG score be used in prospective studies of therapy for MG. A clinically meaningful improvement in a patient's QMG would be a 5 point or greater reduction in score after 26 weeks of treatment.

TABLE 2
Quantitative MG (QMG) Score for Disease Severity

| | Test Items Weakness | | | |
|---|---|---|---|---|
| | None | Mild | Moderate | Severe |
| Grade | 0 | 1 | 2 | 3 |
| Double vision | >60 | 11-60 | 1-20 | Spontaneous |
| (lateral gaze) Sec. | | | | |
| Ptosis (upward | >60 | 11-60 | 1-10 | Spontaneous |
| gaze) Sec. | | | | |
| Facial Muscles | Normal lid closure | Complete, weak, | Complete, without | Incomplete |
| | | some resistance | resistance | |
| Swallowing 4 oz | Normal | Minimal coughing | Severe | Cannot swallow |
| water (½ cup) | | or throat clearing | coughing/choking or | (test not attempted) |
| | | | nasal regurgitation | |
| Speech following | None at #50 | Dysarthria at #30- | Dysarthria at #10- | Dysarthria at #9 |
| counting aloud from | | 49 | 29 | |
| 1-50 (onset of | | | | |
| dysarthria) | | | | |
| Right arm | 240 | 90-239 | 10-89 | 0-9 |
| outstretched (90° | | | | |
| sitting) Sec. | | | | |
| Left arm | 240 | 90-239 | 10-89 | 0-9 |
| outstretched (90° | | | | |
| sitting) Sec. | | | | |
| Forced vital | ≥80% | 65-79% | 50-64% | <50% |
| capacity | | | | |
| Right hand grip (kg) | | | | |
| male | ≥45 | 15-44 | 5-14 | 0-4 |
| female | ≥30 | 10-29 | 5-9 | 0-4 |
| Left hand grip (kg) | | | | |
| male | ≥35 | 15-34 | 5-14 | 0-4 |
| female | ≥25 | 10-24 | 5-9 | 0-4 |
| Head, lifted (45%, | 120 | 30-119 | 1-29 | 0 |
| supine) Sec. | | | | |
| Right leg | 100 | 31-99 | 1-30 | 0 |
| outstretched (45- | | | | |
| 50%, supine) Sec. | | | | |
| Left leg | 100 | 31-99 | 1-30 | 0 |
| outstretched (45- | | | | |
| 50%, supine) Sec. | | | | |

The NeuroaQoL™ Fatigue is a reliable and validated brief 19-item survey of fatigue completed by the subject or patient. Higher scores indicate greater fatigue and greater impact of MG on activities (Table 3; Gershon, R. et al., Oual. Life Res., 2 1:475-86, 2012). A clinically meaningful improvement in a patient's Neuro-QoL™ Fatigue score is reflected in a decrease in score after 26 weeks of treatment.

TABLE 3
Neuro-QoL ™ Fatigue

| | In the past 7 days . . . | Never | Rarely | Sometimes | Often | Always |
|---|---|---|---|---|---|---|
| NQFTG13 | I felt exhausted | 1 | 2 | 3 | 4 | 5 |
| NQFTG11 | I felt that I had no energy | 1 | 2 | 3 | 4 | 5 |
| NQFTG15 | I felt fatigued | 1 | 2 | 3 | 4 | 5 |
| NQFTG06 | I was too tired to do my household | 1 | 2 | 3 | 4 | 5 |
| | chores | | | | | |
| NQFTG07 | I was too tired to leave the house | 1 | 2 | 3 | 4 | 5 |
| NQFTG10 | I was frustrated by being too tired | 1 | 2 | 3 | 4 | 5 |
| | to do the things I wanted to do | | | | | |
| NQFTG14 | I felt tired | 1 | 2 | 3 | 4 | 5 |
| NQFTG02 | I had to limit my social activity | 1 | 2 | 3 | 4 | 5 |
| | because I was tired | | | | | |
| NQFTG01 | I needed help doing my usual | 1 | 2 | 3 | 4 | 5 |
| | activities because of my fatigue | | | | | |
| NQFTG03 | I needed to sleep during the day | 1 | 2 | 3 | 4 | 5 |
| NQFTG04 | I had trouble starting things | 1 | 2 | 3 | 4 | 5 |
| | because I was too tired | | | | | |
| NQFTG05 | I had trouble finishing things | 1 | 2 | 3 | 4 | 5 |
| | because I was too tired | | | | | |
| NQFTG08 | I was too tired to take a short walk | 1 | 2 | 3 | 4 | 5 |
| NQFTG09 | I was too tired to eat | 1 | 2 | 3 | 4 | 5 |
| NQFTG12 | I was so tired that I needed to rest | 1 | 2 | 3 | 4 | 5 |
| | during the day | | | | | |
| NQFTG16 | I felt weak all over | 1 | 2 | 3 | 4 | 5 |
| NQFTG17 | I needed help doing my usual | 1 | 2 | 3 | 4 | 5 |
| | activities because of weakness | | | | | |
| NQFTG18 | I had to limit my social activity | 1 | 2 | 3 | 4 | 5 |
| | because I was physically weak | | | | | |
| NQFTG20 | I had to force myself to get up and | 1 | 2 | 3 | 4 | 5 |
| | do things because I was physically | | | | | |
| | too week | | | | | |

Subjects with increasingly severe MG can suffer from potentially fatal respiratory complications including profound respiratory muscle weakness. Respiratory function is monitored closely for evidence of respiratory failure in MG subjects and ventilator support is recommended in the event of consistent declines in serial measurements of Forced Vital Capacity (FVC) or NIF, loss of upper airway integrity (difficulty handling oral secretions, swallowing, or speaking) or in the setting of emerging respiratory failure. FVC, as one of the test items in QMG, is performed when QMG is performed. NIF is performed using the NIF Meter.

The MG clinical state is assessed using the MGFA Post-intervention Status (MGFA-PIS).

Change in status categories of “Improved,” “Unchanged,” “Worse,” “Exacerbation,” and “Died of MG” as well as the Minimal Manifestations (MM) can be assessed (Table 4).

TABLE 4
MGFA-PIS

| | |
|---|---|
| Complete Stable | The patient has no symptoms r signs of MG for at least 1 year and has received no |
| Remission (CSR) | therapy for MG during that time. There is no weakness of any muscle on careful |
| | examination by someone skilled in the evaluation of neuromuscular disease. Isolated |
| | weakness of eyelid closure is accepted. |
| Pharmacologic | The same criteria as for CSR except that the patient continues to take some form of |
| Remission (PR) | therapy for MG. Patients taking cholinesterase inhibitors are excluded from this |
| | category because their use suggests the presence of weakness. |
| Minimal Manifestations | The patient has no symptoms of functional limitations from MG but has some |
| (MM) | weakness on examination of some muscles. This class recognizes that some patients |
| | who otherwise meet the definition of CSR or PR do have weakness that is only |
| | detectable by careful examination. |
| MM-0 | The patient has received no MG treatment for at least 1 year. |
| MM-1 | The patient continues to receive some form of immunosuppression but no |
| | cholinesterase inhibitors or other symptomatic therapy. |
| MM-2 | The patient has received only low-dose cholinesterase inhibitors (<120 mg |
| | pyridostigmine/day) for at least 1 year. |
| MM-3 | The patient has received cholinesterase inhibitors or other symptomatic therapy and |
| | some form of immunosuppression during the past year. |
| Change in Status | |
| Improved (I) | A substantial decrease in pretreatment clinical manifestations or a sustained |
| | substantial reduction in MG medications as defined in the protocol. In prospective |
| | studies, this should be defined as a specific decrease in QMG score. |
| Unchanged (U) | No substantial change in pretreatment clinical manifestations or reduction in MG |
| | medications as defined in the protocol. In prospective studies, this should be defined |
| | in terms of a maximum change in QMG score. |
| Worse (W) | A substantial increase in pretreatment clinical manifestations or a substantial increase |
| | in MG medications as defined in the protocol. In prospective studies, this should be |
| | defined as a specific increase in QMG score. |
| Exacerbation (B) | Patients who have fulfilled criteria of CSR, PR, or MM but subsequently developed |
| | clinical findings greater than permitted by these criteria. |
| Died of MG (D of MG) | Patients who died of MG, of complications of MG therapy, or within 30 days after |
| | thymectomy. List the cause (see Morbidity and Mortality table). |

In some embodiments, a subject administered Compound 1 or a pharmaceutically acceptable PG 25T salt thereof shows a reduced MG-ADL score. In some embodiments, the subjects has an initial MG-ADL score of greater than 6 points. In some embodiments, the subject has an initial MG-ADL score greater than 0, 1, 2, 3, 4, 5, 6, 7, 8, 9, 10, 11, 12, 13, 14, 15, 16, 17, 18, 19, 20, 21, 22 or 23 points. In some embodiments, after a course of treatment with Compound 1 or a pharmaceutically acceptable salt, the MG-ADL score of the subject is reduced to less than 6 points. In some embodiments, the MG-ADL score is reduced by at least 1 point, at least 2 points, at least 3 points, at least 4 points, at least 5 points, at least 6 points, at least 7 points, at least 8 points, at least 9 points, at least 10 points, at least 11 points, at least 12 points, at least 13 points, at least 14 points, at least 15 points, at least 16 points, at least 17 points, at least 18 points, at least 19 points, at least 20 points, at least 21 points, at least 22 points, at least 23 points, or at least 24 points after treatment with Compound 1 or a pharmaceutically acceptable salt thereof. In some embodiments, the MG-ADL score of the subject is reduced by at least 1 point after a course of treatment with Compound 1 or a pharmaceutically acceptable salt thereof. In some embodiments, the MG-ADL score of the subject is reduced by 1, 2, 3, 4, 5, 6, 7, 8, 9, 0, 11, 12, 13, 14, 15, 16, 17, 18, 19, 20, 21, 22, 23, or 24 points after a course of treatment with Compound 1 or a pharmaceutically salt thereof.

In some embodiments, the course of treatment with Compound 1 or a pharmaceutically acceptable salt thereof lasts for 26 weeks. In some embodiments, the course of treatment lasts for 26-52, 26-78, 26-104, 26-130, 26-156, 26-182, 26-208 weeks, or more. In some embodiments, the course of treatment lasts for greater than 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156 or 182 weeks. In some embodiments, the course of treatment lasts for greater than 1, 2, 3, 4, 5, 10, 15, 20, 25, 30, 35, 40, 45, 50, 55, 60, 65, 70, 75, 80, or more years. In some embodiments, the course of treatment lasts for the remainder of the subject's life.

In some embodiments, one or more symptoms or scores associated with MG improves during the course of treatment and is maintained at the improved level throughout treatment. The MG-ADL score can improve, for example, after 26 weeks of treatment with Compound 1 or a pharmaceutically acceptable salt thereof and then remain at the improved level for the duration of the treatment (e.g., 52 weeks).

In some embodiments, the first sign of improvement occurs by 26 weeks of treatment with Compound 1 or a pharmaceutically acceptable salt thereof. In some embodiments, the first sign of improvement occurs between weeks 1-26, 26-52, 52-78, 78-104, 104-130, 130-156, 156-182, or 182-208 of treatment with Compound 1 or a pharmaceutically acceptable salt thereof. In some embodiments, the first sign of improvement occurs at week 1, 2, 3, 4, 5, 6, 7, 8, 9, 10, 11, 12, 13, 14, 15, 16, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156 or 182.

In some embodiments, the MG is gMG. In some embodiments, the gMG is refractory gMG. In some embodiments, a subject suffering from refractory gMG is characterized as a subject who is positive for auto-antibodies binding to AChR (anti-AChR antibodies) who continues to show marked generalized weakness or bulbar signs and symptoms of MG while receiving current standard of care for MG such as cholinesterase inhibitor therapy and IST or who requires chronic plasma exchange or chronic IVIg to maintain clinical stability. In some embodiments, a subject suffering from refractory gMG is characterized as one who continues to show marked generalized weakness or bulbar signs and symptoms of MG while receiving current standard of care for MG such as cholinesterase inhibitor therapy and IST or who requires chronic plasma exchange or chronic IVIg to maintain clinical stability.

### Pharmaceutical Compositions

The disclosure also relates to use of pharmaceutical compositions comprising Compound 1 and or a pharmaceutically acceptable salt thereof. Any suitable pharmaceutical compositions and formulations, as well as suitable methods for formulating and suitable routes and suitable sites of administration, are within the scope of this disclosure. Also, unless otherwise stated, any suitable dosage(s) and frequency of administration are contemplated.

Unless otherwise noted, the dosage level of Compound 1 or a pharmaceutically acceptable salt thereof can be any suitable level. In some embodiments, the dosage levels of Compound 1 or a pharmaceutically acceptable salt thereof for a subject can generally be between about 1 mg/kg and about 100 mg/kg (e.g., between about 2 mg/kg and about 50 mg/kg, between about 5 mg/kg and about 25 mg/kg), per treatment.

The compositions can be administered to a human subject using a variety of methods that depend, in part, on the route of administration. The route can be, e.g., oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration.

In some embodiments, a composition is formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Compositions for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In some embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, di-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which compositions can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

In some embodiments, the oral dosage form, such as a solution or suspension formed by mixing a triturated tablet or crystal or a powder with water, can be administered via a nasogastric tube.

A suitable dose of Compound 1 or a pharmaceutically acceptable thereof which is capable of treating MG in a subject, can depend on a variety of factors including, e.g., the age, gender, and weight of a subject to be treated and the particular inhibitor compound used. Other factors affecting the dose administered to the subject include, e.g., the type or severity of MG. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will depend upon the judgment of the treating medical practitioner (e.g., doctor or nurse). A pharmaceutical composition can include a therapeutically effective amount of Compound 1 or a pharmaceutically acceptable salt thereof. Such effective amounts can be readily determined by one of ordinary skill in the art.

### Kits and Unit Dosage Forms

Also provided herein are kits that include Compound 1 or a pharmaceutically acceptable salt thereof in a therapeutically effective amount (e.g., in a pharmaceutical composition) for use in any one or more of the methods disclosed herein. The kit may optionally include instructions, e.g., comprising administration schedules, to allow a practitioner (e.g., a physician, nurse, or patient) to administer Compound 1 or the pharmaceutically acceptable salt thereof e.g., in a pharmaceutical composition further including a pharmaceutically acceptable carrier) contained therein to a patient having MG. The kit may further include a syringe.

Kits can optionally include multiple packages of the single-dose pharmaceutical compositions each containing an effective amount of Compound 1 or a pharmaceutically acceptable salt thereof e.g., in a pharmaceutical composition) for a single administration in accordance with the methods provided above. Instruments or devices for administering Compound 1 or a pharmaceutically acceptable salt thereof (e.g., in a pharmaceutical composition) may also be included in the kits. A kit may provide one or more pre-filled syringes containing an effective amount of Compound 1 or a pharmaceutically acceptable salt thereof e.g., in a pharmaceutical composition).

The following examples are merely illustrative and should not be construed as limiting the scope of this disclosure in any way as many variations and equivalents will become apparent to those skilled in the art upon reading the present disclosure. The contents of all references, accessioned entries (e.g., PUBMED, GENBANK, UNIPROT, PUBCHEM entries), patents, and patent applications cited throughout this application are expressly incorporated herein by reference.

### Examples

### Example 1. A Phase 2, Randomized, Double-Blind, Placebo-Controlled, Multicenter Study to

Evaluate the Efficacy and Safety of Compound 1 (ALXN2050; vemircopan) in Adult Participants with Generalized Myasthenia Gravis A Phase 2, randomized, double-blind, placebo-controlled, multicenter study is conducted to evaluate the safety and efficacy of orally administered Compound 1 (ALXN2050; vemircopan) in adult patients.

### Objectives and Endpoints

The primary objective of the study is to assess the efficacy of ALXN2050 compared with placebo in the treatment of generalized MG (gMG) based on the improvement in the Myasthenia Gravis Activities of Daily Living (MG-ADL) total score. The secondary objectives of this study are to assess the efficacy of ALXN2050 compared with placebo in the treatment of gMG based on (1) the improvement in the Quantitative Myasthenia Gravis (QMG) total score, (2) the improvement in quality-of-life measures, and (3) additional endpoints involving the MG-ADL total score.

Other objectives of this study are to (1) characterize the PK/PD of ALXN2050 and to establish the PK/PD relationship in participants with gMG, (2) assess the effect of factor D inhibition on complement biomarkers, (3) characterize the overall safety of ALXN2050 compared with placebo in participants with gMG, (4) assess the efficacy of ALXN2050 compared with placebo in the treatment of gMG based on other efficacy endpoints, (5) assess the effect of factor D inhibition on ACHR antibody titers, and (6) characterize non-genetic biomarkers in adult participants with gMG.

The objectives and corresponding endpoints are summarized in Table 5 below.

TABLE 5
Study Objectives and Endpoints

| | |
|---|---|
| Objectives | Endpoints |

Primary

| | |
|---|---|
| To assess the efficacy of ALXN2050 compared | Proportion of participants with an MG-ADL total |
| with placebo in the treatment of generalized MG | score reduction of ≥2 points in any 4 consecutive |
| (gMG) based on improvement in the Myasthenia | weeks during the first 8 weeks and who did not |
| Gravis Activities of Daily Living (MG-ADL) total | receive rescue therapy |
| score | |

Secondary

| | |
|---|---|
| To assess the efficacy of ALXN2050 compared | Change from baseline in QMG total score at |
| with placebo in the treatment of gMG based on | Week 8 |
| improvement in the Quantitative Myasthenia Gravis | Proportion of participants with at least a 3-, 4-, |
| (QMG) total score | 5-, 6-, 7-, or 8-point improvement in the QMG |
| | total score at Week 8 |
| | Proportion of participants with at least a 3-, 4-, |
| | 5-, 6-, 7-, or 8-point improvement in the QMG |
| | total score in any 4 consecutive weeks during |
| | the first 8 weeks and who did not receive |
| | rescue therapy |
| To assess the efficacy of ALXN2050 compared | Change from baseline in Neuro-QoL ™ Fatigue |
| with placebo in the treatment of gMG based on | score at Week 8 |
| improvement in quality of life measures | |
| To assess the efficacy of ALXN2050 compared | Change from baseline in MG-ADL total score at |
| with placebo in the treatment of gMG based on | Week 8 |
| additional endpoints involving the MG-ADL total | Proportion of participants with at least a 3-, 4-, |
| score | 5-, 6-, 7-, or 8-point improvement in the |
| | MG-ADL total score in any 4 consecutive |
| | weeks during the first 8 weeks and who did not |
| | receive rescue therapy |
| | Proportion of participants with at least a 2-, 3-, |
| | 4-, 5-, 6-, 7-, or 8-point improvement in the MG- |
| | ADL total score at Week 8 |

PK/PD

| | |
|---|---|
| To characterize the PK/PD of ALXN2050 and to | Observed Cₘₐₓ and C_{trough} values over time |
| establish the PK/PD relationship in participants with | Absolute values and change from baseline in |
| gMG | plasma Bb concentration and serum AP activity |
| | over time |

Biomarker

| | |
|---|---|
| To assess the effect of factor D inhibition on | Plasma factor D concentration, serum C3 |
| complement biomarkers | concentration, and serum CP activity over time |

Safety

| | |
|---|---|
| To characterize the overall safety of ALXN2050 | Incidence of TEAEs and TESAEs over time |
| compared with placebo in participants with gMG | Changes from baseline in laboratory |
| | assessments |

Tertiary/Exploratory

| | |
|---|---|
| To assess the efficacy of ALXN2050 compared | Proportion of participants with an MG-ADL total |
| with placebo in the treatment of gMG based on | score reduction of ≥2 points and a QMG total |
| other efficacy endpoints | score reduction of ≥3 points in any |
| | 4 consecutive weeks during the first 8 weeks |
| | and who did not receive rescue therapy |
| | Proportion of participants with at least a 2-point |
| | improvement in the MG-ADL total score for |
| | 4 consecutive weeks (measured from Day 1 to |
| | Week 8 for Groups 1, 2, and 3 and from |
| | Week 8 to Week 16 for Groups 3a and 3b) and |
| | who did not receive any rescue therapy |
| | Change from baseline in MG-ADL total score at |
| | Week 8 for Groups 1, 2, and 3; and from |
| | Week 8 to Week 16 for Groups 3a and 3b |
| | Change from baseline in MG-ADL total score at |
| | Week 26 for Groups 1 and 2^{a} |
| | Change from baseline in MG-ADL total score at |
| | Week 26 for Groups 1 and 2Error! Reference |
| | source not found.; and from Week 8 to Week 34 |
| | for Groups 3a and 3bError! Reference source |
| | not found. |
| | Incidence of Clinical Deterioration of gMG over |
| | time^{b} |
| | MGFA-PIS at Week 8 and Week 26Error! |
| | Reference source not found. |
| | Proportion of participants with a classification |
| | of Minimal Manifestations at Week 8 and |
| | Week 26Error! Reference source not found. (as |
| | measured by the MGFA-PIS) |
| | Proportion of participants who receive rescue |
| | therapy over time |
| To assess the effect of factor D inhibition on AChR | Change of anti-AChR antibody titers over time |
| antibody titers | |
| To characterize nongenetic biomarkers in adult | Detection of gMG-associated autoantibodies, |
| participants with gMG | which may include baseline and/or later |
| | timepoints (eg, MuSK, LRP4) |
| | In vitro evaluation of autoantibody activity |
| | (eg, AChR blocking, complement deposition) |
| | Absolute values and change from baseline in |
| | levels of complement proteins and complement |
| | pathway regulators (eg, C5b-9, Properdin) |
| | Change from baseline in biomarkers of |
| | inflammation and NMJ damage (eg, MMP-10, |
| | IL-6) |

Error! Reference source not found. The comparator will be discussed in detail in the Statistical Analysis Plan.
Error! Reference source not found. Clinical Deterioration is as defined herein.
Abbreviations: AChR = acetylcholine receptor; AP = alternative pathway; Bb = complement factor Bb; C3 = complement component 3; C5 = complement component 5; Cₘₐₓ = maximum (peak) plasma concentration of the drug; CP = classical pathway; C_{trough} = pre-dose concentration; IL = interleukin; LRP4 = low density lipoprotein receptor-related protein 4; MG = myasthenia gravis; MGFA-PIS = Myasthenia Gravis Foundation of America Post-Intervention Status; MMP = matrix metalloproteinase; MuSK = muscle-specific tyrosine kinase; Neuro-QoL ™ Fatigue = Quality of Life in Neurological Disorders Fatigue questionnaire; NMJ = neuromuscular junction; PD = pharmacodynamic; PK = pharmacokinetic; TEAE = treatment-emergent adverse event; TESAE = treatment-emergent serious adverse event

### Overall Design

This is a Phase 2, randomized, double-blind, parallel-group, placebo-controlled, multicenter study to evaluate the efficacy and safety of ALXN2050 in adult participants with gMG. Approximately 70 eligible participants will be stratified by MG-ADL total score at baseline (≤7 versus >7) and randomized on Day 1 in a 2:1:2 ratio to 1 of 3 treatment groups: ALXN2050 180 mg bid (Group 1), ALXN2050 120 mg bid (Group 2), or placebo (Group 3).

Participants will receive the study intervention bid from Day 1 through Week 117. Participants may continue to receive a stable regimen of acetylcholinesterase inhibitors (AChEIs), supportive IST (with some exceptions as defined herein), and/or corticosteroid therapy that was being administered prior to the Screening Visit, but no new AChEls/ISTs/steroids and no change in AChEI/IST/steroid dosages are permitted during the Screening Period, the PEP, or the ETP, except for safety reasons as identified by the Investigator. During the OLE Period (i.e., after 34 weeks of treatment), changes in supportive AChEls/ISTs/steroids can be made at the discretion of the Investigator.

Rescue therapy (e.g., plasmapheresis (PP)/plasma exchange (PE), intravenous immunoglobulin [IVIg], or high-dose corticosteroid) is allowed at any time for participants who experience a protocol-defined Clinical Deterioration (as defined herein). The treatment approach for a specific participant should be determined by the Investigator.

Multiple outcome measures (including participant-reported outcome (PRO) and clinician-reported outcome (ClinRO); Table 6) will be administered to evaluate the efficacy and safety objectives. In the clinic, assessments should be performed or administered by a properly trained Clinical Evaluator (e.g., neurologist, neurologist in-training, or delegated member of the investigational site staff).

TABLE 6
Assessments Administered During the Study

| Study Assessment/Questionnaires | Responsible Study Staff | Type of Assessment |
|---|---|---|
| C-SSRS | Clinical Evaluator | PRO |
| MGFA classification | Investigator or neurologist | NA |
| MG-ADL | Clinical Evaluator | ClinRO |
| MGFA-PIS | Investigator or neurologist | ClinRO |
| QMG | Clinical Evaluator | ClinRO |
| Neuro-QoL ™ Fatigue | NA | PRO |

Abbreviations: ClinRO = clinician-reported outcome; C-SSRS = Columbia-Suicide Severity Rating Scale; MG-ADL = Myasthenia Gravis Activities of Daily Living profile; MGFA = Myasthenia Gravis Foundation of America; MGFA-PIS = Myasthenia Gravis Foundation of America Post-Intervention Status; NA = not applicable; Neuro-QoL ™ Fatigue = Neurological Quality of Life Fatigue questionnaire; PRO = participant-reported outcome; QMG = Quantitative Myasthenia Gravis score

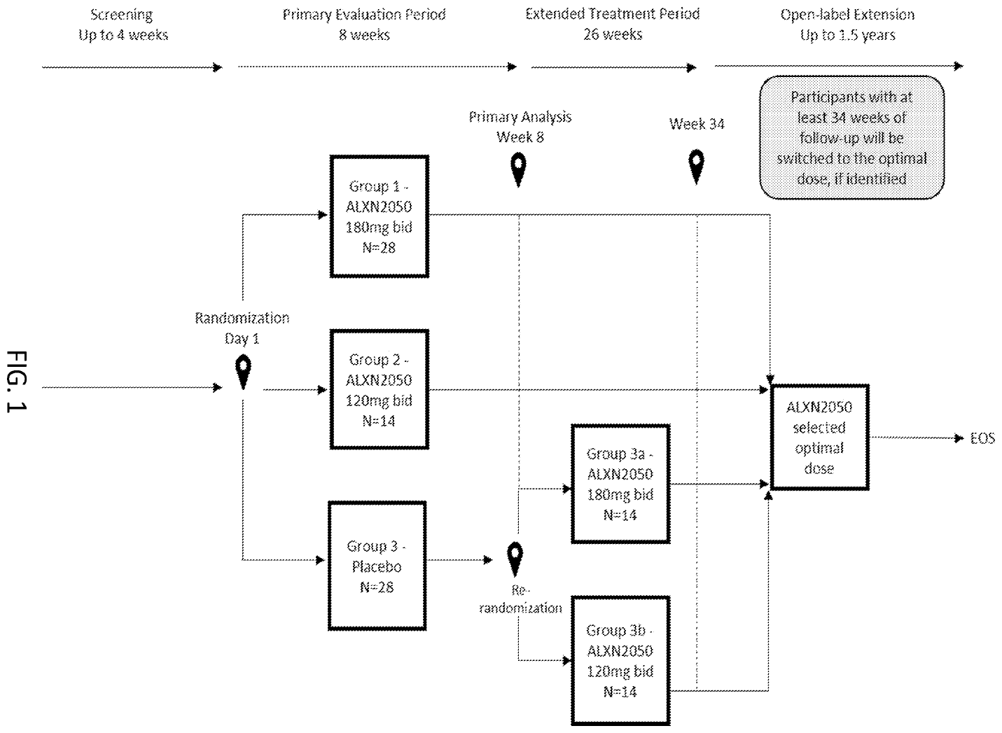

There will be 4 periods in this study (FIG. 1):

### Screening Period of Up to 4 Weeks

During this timeframe, participants will present to the clinic at least once wherein screening procedures and assessments as indicated in the Schedule of Activities (SoA; Table 7) will be performed to determine eligibility. The participant may be asked to return to the clinic for additional follow-up (e.g., blood redraw), although it is not anticipated that this will be a frequent occurrence.

Primary Evaluation Period of 8 weeks (Day 1 to Week 8)

Treatment during this timeframe will be blinded for participants, Investigators, site personnel, and Alexion staff. The participant will present to the clinic every week for the first 8 weeks for fulfillment of procedures and assessments as specified in the SoA (Table 7). For the first visit on Day 1, and after confirmation of eligibility, the participant will be randomized to 1 of 3 treatment groups. The participant will be dispensed a container of the study intervention that will, at a minimum, cover bid dosing for 30 days. In the clinic, the participant will be administered a dose of study intervention (3 tablets). The participant will be instructed to take the second dose of study intervention (3 tablets) at home. The participant will also be dispensed the safety card that discusses some of the risks associated with treatment, and steps to take in the event of an emergency. The participant must be instructed to carry the safety card at all times. The last visit for the PEP will be the Week 8 Visit.

Extended Treatment Period of 26 weeks (Week 8 to Week 34)

During this timeframe, participants in the ALXN2050 180 mg BID and ALXN2050 120 mg BID groups will continue taking the dose to which they are randomized. Participants in the placebo group will be stratified by MG-ADL total score at baseline (pre-placebo) and re-randomized in a 1:1 ratio to either ALXN2050 180 mg BID (Group 3a) or ALXN2050 120 mg BID (Group 3b). All participants will receive active study intervention; however, the actual dosage of ALXN2050 will be blinded to participants, Investigators, and site personnel. Visits at Weeks 9, 10, 11, 13, 14, and 15 will be conducted via telephone contact (Table 8). Participants will present for in-clinic visits at Weeks 12, 16, 26, and 34. The first dose of study intervention during in-clinic visit days will be administered by the site personnel. The participant will take the second dose of study intervention at home. The last visit for the ETP will be the Week 34 Visit.

Open-label Extension Period of up to approximately 1.5 years (Week 34 to Week 117) During this timeframe, all participants will receive active study intervention. If an optimal dose of ALXN2050 has been identified, all participants will be switched to this optimal dose, as long they have completed the first 34 weeks of treatment. However, the actual dose of ALXN2050 will continue to be blinded to participants, Investigators, and site personnel during this study period until they are switched to the optimal dose, if one is identified during the study. Participants will return to the clinic approximately every 3 months as indicated in the SoA (Table 9). The first dose of study intervention during in-clinic visit days will be administered by the site personnel. The participant will take the second dose of study intervention at home. The last visit for the OLE Period is the EOS Visit.

An EOS Visit will occur 30 (±2) days after the last dose of study intervention for all participants. The overall study duration for an individual participant will be approximately 125 weeks (from the Screening Visit through the EOS Visit).

TABLE 7
Schedule of Activities (Screening and Primary Evaluation Periods)

| | Period | | | | | | | | | | | | |
|---|---|---|---|---|---|---|---|---|---|---|---|---|---|
| | Screening | | | | | | | | | | | | |
| | Period | Primary Evaluation Period | | | | | | | | | | | |
| | Study Day (D) | | | | | | | | | | | | |
| | Up to 27 | | | | | | | | | | | | |
| | days prior | | | | | | | | | | | | |
| | to D1 | D 1 | D 8 | D 15 | D 22 | D 29 | D 36 | D 43 | D 50 | D 57 | ED^{a} | CD^{b} | EOS^{a} |
| | Study Week (W) | | | | | | | | | | | | |
| | NA | NA | W 1 | W 2 | W 3 | W 4 | W 5 | W 6 | W 7 | W 8 | NA | NA | NA |
| | Study Window (days) | | | | | | | | | | | | |
| | NA | NA | ±1 | ±1 | ±1 | ±1 | ±1 | ±1 | ±1 | ±1 | NA | NA | ±2 |
| Eligibility | | | | | | | | | | | | | |
| Informed consent | X | | | | | | | | | | | | |
| Inclusion/exclusion | X | X^{c} | | | | | | | | | | | |
| Medical history^{d} | X | | | | | | | | | | | | |
| MG history^{e} | X | | | | | | | | | | | | |
| MGFA classification^{f} | X | | | | | | | | | | | | |
| Height | X | | | | | | | | | | | | |
| Weight | X | | | | | | | | | | | | |
| Demographics | X | | | | | | | | | | | | |
| Study Administrative | | | | | | | | | | | | | |
| Vaccination or | X | | | | | | | | | | | | |
| confirmation of | | | | | | | | | | | | | |
| vaccination against | | | | | | | | | | | | | |
| Neisseria meningitidis^{g} | | | | | | | | | | | | | |
| Screening | | | | | | | | | | | | | |
| Laboratory Tests | | | | | | | | | | | | | |
| HIV-1, HIV-2, | X | | | | | | | | | | | | |
| Hepatitis B and C | | | | | | | | | | | | | |
| Serum pregnancy^{h} | X | | | | | | | | | | X | | X |
| Urine pregnancy^{h} | | X | | | | X | | | | X | | X | |
| Follicle-stimulating | X | | | | | | | | | | | | |
| hormoneⁱ | | | | | | | | | | | | | |
| Randomization | | | | | | | | | | | | | |
| Randomized to study | | X | | | | | | | | X^{k} | | | |
| intervention^{j} | | | | | | | | | | | | | |
| Study Intervention | | | | | | | | | | | | | |
| Dispensation of study | | X | X | X | X | X | X | X | X | X | | X | |
| intervention^{l} | | | | | | | | | | | | | |
| Administration of | | X | X | X | X | X | X | X | X | X | | X | |
| ALXN2050 or | | | | | | | | | | | | | |
| Placebo^{m} | | | | | | | | | | | | | |
| Efficacy | | | | | | | | | | | | | |
| Assessments | | | | | | | | | | | | | |
| QMG^{f, n} | | X | X | X | X | X | X | X | X | X | X | X | |
| MG-ADL^{f, o} | X | X | X | X | X | X | X | X | X | X | X | X | |
| MGFA-PIS^{f} | | | | | | X | | | | X | X | | |
| Neuro-QoL ™ Fatigue | | X | | | | X | | | | X | X | X | |
| Safety Assessments | | | | | | | | | | | | | |
| C-SSRS Baseline^{p} | | X | | | | | | | | | | | |
| C-SSRS Since Last | | | X | X | X | X | X | X | X | X | X | X | |
| Visit^{p} | | | | | | | | | | | | | |
| Electrocardiogram | X | X | | | | | | | | X | X | | |
| Complete physical | X | | | | | | | | | | X | X | |
| examination, including | | | | | | | | | | | | | |
| neurological | | | | | | | | | | | | | |
| component | | | | | | | | | | | | | |
| Abbreviated physical | | X | X | X | X | X | X | X | X | X | | | |
| examination^{q, r} | | | | | | | | | | | | | |
| Vital sign | X | X | X | X | X | X | X | X | X | X | X | X | |
| measurements^{s} | | | | | | | | | | | | | |
| Adverse event review | X | X | X | X | X | X | X | X | X | X | X | X | X |
| and evaluation | | | | | | | | | | | | | |
| Assess for CD^{b} | X | X | X | X | X | X | X | X | X | X | X | | |
| Review participant | | X | X | X | X | X | X | X | X | X | X | X | |
| safety card^{t} | | | | | | | | | | | | | |
| Safety Laboratory | | | | | | | | | | | | | |
| Tests^{u} | | | | | | | | | | | | | |
| Clinical chemistry | X | X | | | | X | | | | X | X | X | |
| Hematology | X | X | | | | X | | | | X | X | X | |
| Coagulation panel | X | X | | | | X | | | | X | X | X | |
| Urinalysis | X | X | | | | X | | | | X | X | X | |
| PK/PD Tests^{v} | | | | | | | | | | | | | |
| PK | | X^{w} | | | | X^{x} | | | | X^{w} | | X^{x} | |
| PD: AP activity and | | X | | | | X | | | | X | | X | |
| Bb^{y} | | | | | | | | | | | | | |
| Biomarker Tests | | | | | | | | | | | | | |
| Anti-AChR antibody | X | X^{z} | | | | X | | | | X | X | X | |
| Factor D, C3, CP | | X | | | | X | | | | X | X | X | |
| activity, nongenetic | | | | | | | | | | | | | |
| exploratory | | | | | | | | | | | | | |
| biomarkers (eg, | | | | | | | | | | | | | |
| MuSK, LRP4)^{aa} | | | | | | | | | | | | | |
| Other | | | | | | | | | | | | | |
| Concomitant | X | X | X | X | X | X | X | X | X | X | X | X | X |
| medication | | | | | | | | | | | | | |
| Nonpharmacologic | X | X | X | X | X | X | X | X | X | X | X | X | X |
| treatments and | | | | | | | | | | | | | |
| therapies | | | | | | | | | | | | | |

^{a}If a participant is discontinued from the study during the PEP, an ED Visit will be performed, and then an EOS Visit will be performed 30 (±2) days after the last dose of study intervention.
^{b}Evaluation of CD must be performed as soon as possible, within 48 hours, of notification to the Investigator of symptom onset. If CD occurs between scheduled visits, only the assessments for the CD Visit are needed. If CD occurs on a scheduled visit, all scheduled assessments should be performed for that visit as well as for the evaluation of CD. Additional evaluation visits may be scheduled at the discretion of the Investigator.
^{c}Confirm on Day 1.
^{d}Includes substance usage, and past and current medical conditions, including surgical history.
^{e}The participant's MG history and relevant medical history, including prior and concomitant conditions/disorders, treatment history, substance usage, and history of medical conditions and surgeries will be evaluated by the Investigator and documented in the source documents and eCRF. Myasthenia gravis history will include diagnosis date; initial MG clinical presentation (ocular myasthenia gravis [oMG] or gMG); time to gMG, if initial clinical presentation was oMG; maximum MGFA classification since diagnosis; ventilatory support since diagnosis; dates of MG exacerbation or crisis since diagnosis and prior to Day 1; and any MG-related hospitalizations within 2 years prior to the Screening Visit. Myasthenia gravis-specific medication or therapy taken within 2 years prior to the Screening Visit should also be recorded.
^{f}MG assessments should be performed at approximately the same time of day by a properly trained Clinical Evaluator (preferably the same evaluator) throughout the study. The MG-ADL should always be performed first, followed by the QMG.
Note:
A Clinical Evaluator may be a neurologist, neurologist in training, or delegated member of the investigational site staff who has been certified in administering the assessments.
^{g}To reduce the risk of meningococcal infection (Neisseria meningitidis), all participants must be vaccinated against meningococcal infection within 3 years or before the administration of study intervention on Day 1. Participants who initiate study intervention treatment less than 2 weeks after receiving a meningococcal vaccine must receive treatment with appropriate prophylactic antibiotics until at least 2 weeks after vaccination.
^{h}Pregnancy tests must be performed on all participants of childbearing potential at the specified time points. Serum pregnancy test will be performed at the Screening, ED, and EOS visits; urine pregnancy tests will be performed locally at all other specified time points. Additional pregnancy tests (urine or serum) may also be performed at any in-clinic visit at the Investigator's discretion.
ⁱFollicle-stimulating hormone may be obtained at the Screening Visit to confirm postmenopausal status in female participants who are considered postmenopausal ONLY. This test is not needed for men and will not be conducted in women of childbearing potential.
^{j}All participants who continue to meet all inclusion criteria and none of the exclusion criteria and have been cleared for randomization by the Investigator will be centrally randomized via an interactive response technology.
^{k}Participants randomized to placebo will be further randomized to 1 of the 2 active treatment groups (i.e., ALXN2050 120 or 180 mg bid).
^{l}Ensure kit and lot number are recorded on the drug accountability log.
^{m}Dosing is by mouth twice daily throughout the PEP.
ⁿThe QMG assessment should be performed by a properly trained Clinical Evaluator (preferably the same evaluator) throughout the study. If a participant is taking a cholinesterase inhibitor, the dose must be withheld for at least 8-12 hours prior to the assessment and, whenever possible, the time from the last dose to the QMG assessment should be kept similar between visits.
^{o}The MG-ADL is required to be performed first, followed by the QMG. The MG-ADL assessment should be performed by a properly trained Clinical Evaluator (preferably the same evaluator) throughout the study. The recall period for MG-ADL is the preceding 7 days or since the last visit if the visit interval is less than 7 days.
^{p}C-SSRS will be assessed for both lifetime and past 12 months at Baseline, and Since Last Visit for subsequent visits.
^{q}The abbreviated physical examination will be performed, if necessary, on the basis of the participant's health status and the clinical judgment of the Investigator.
^{r}A symptom based neurologic examination should be performed if the participant has any complaints or clinical findings attributable to the central nervous system and if positive for findings, a full neurologic examination will need to be performed at that assessment time point and at future time points as needed (determined by the Investigator).
^{s}Vital sign measurements will include systolic and diastolic blood pressure (millimeters of mercury [mmHg]), heart rate (beats/minute), and temperature (degrees Celsius [° C.] or degrees Fahrenheit [° F.]). On dosing days, vital signs will be taken before study intervention administration and after the participant has been resting for at least 5 minutes.
^{t}Participants will be given a safety card prior to the first dose of study intervention. At each visit throughout the study, investigational site staff will ensure that the participant has the safety card and will review the guidelines with the participant.
^{u}Safety laboratory samples will be analyzed by the central laboratory. A list of parameters that will be obtained during the study are provided in Table 10.
^{v}Baseline and trough blood samples for serum PK will be collected predose (within 30 minutes prior to the administration of study intervention). Peak blood samples for serum PK/PD are to be taken 2 and 4 hours postdose. All collection times will be recorded in the participant's electronic case report form.
^{w}Trough (within 30 minutes predose) and peak (2 hours and 4 hours postdose).
^{x}Trough (within 30 minutes predose).
^{y}AP activity within 30 minutes predose and 2 and 4 hours postdose at Day 1 and Week 8, and within 30 minutes predose at Week 4 and CD. Bb sampling within 30 minutes predose and 2 hours postdose at Day 1, and within 30 minutes predose at all other in clinic visits.
^{z}Collect predose on Day 1.
^{aa}Collect predose.
Abbreviations: AChR = acetylcholine receptor; AP = alternative pathway; bid = twice daily; Bb = fragment of complement factor B; C3 = complement component 3; CD = Clinical Deterioration; CP = classical pathway; C-SSRS = Columbia Suicide Severity Rating Scale; D = Day; ED = early discontinuation; EOS = end of study; gMG = generalized myasthenia gravis; HIV = human immunodeficiency virus; IL = interleukin; LRP4 = low density lipoprotein receptor-related protein 4; MG = myasthenia gravis; MG-ADL = Myasthenia Gravis Activities of Daily Living profile; MGFA = Myasthenia Gravis Foundation of America; MGFA-PIS = Myasthenia Gravis Foundation of America Post-Intervention Status; MMP = matrix metalloproteinase; MuSK = muscle-specific tyrosine kinase; Neuro-QOL ™ Fatigue = Quality of Life in Neurological Disorders Fatigue questionnaire; oMG = ocular myasthenia gravis; PD = pharmacodynamic; PEP = Primary Evaluation Period; PK = pharmacokinetic; QMG = Quantitative Myasthenia Gravis score for disease severity; W = Week

TABLE 8
Schedule of Activities (Extended Treatment Period)

| | Period | | | | | | | | | | | | |
|---|---|---|---|---|---|---|---|---|---|---|---|---|---|
| | Extended Treatment Period^{a} | | | | | | | | | | | | |
| | Study Year | | | | | | | | | | | | |
| | Year 1 | | | | | | | | | | | | |
| | Study Day (D) | | | | | | | | | | | | |
| | D64 | D 71 | D 78 | D 85 | D 92 | D 99 | D 106 | D 113 | D 183 | D 239 | ED^{b} | CD^{c} | EOS^{b} |
| | Study Week (W) | | | | | | | | | | | | |
| | W 9 | W 10 | W 11 | W 12 | W 13 | W 14 | W 15 | W 16 | W 26 | W 34 | NA | NA | NA |
| | Visit Type | | | | | | | | | | | | |
| | Phone | Phone | Phone | In- | Phone | Phone | Phone | In- | In- | In- | In- | In- | In- |
| | call | call | call | clinic | call | call | call | clinic | clinic | clinic | clinic | clinic | clinic |
| | Study Window (days) | | | | | | | | | | | | |
| | NA | NA | NA | ±3 | NA | NA | NA | ±3 | ±3 | ±3 | NA | NA | ±2 |
| Administration of | | | | | | | | | | | | | |
| Study Intervention | | | | | | | | | | | | | |
| Dispensation of | | | | X | | | | X | X | X | | X | |
| study intervention^{d} | | | | | | | | | | | | | |
| Administration of | X | X | X | X | X | X | X | X | X | X | | X | |
| ALXN2050^{e} | | | | | | | | | | | | | |
| Efficacy | | | | | | | | | | | | | |
| Assessments | | | | | | | | | | | | | |
| QMG^{f, g} | | | | X | | | | X | X | X | X | X | |
| MG-ADL^{f, h} | X | X | X | X | X | X | X | X | X | X | X | X | |
| MGFA-PIS^{f} | | | | X | | | | X | X | X | X | | |
| Neuro-QoL ™ | | | | X | | | | X | X | X | X | X | |
| Fatigue | | | | | | | | | | | | | |
| Safety | | | | | | | | | | | | | |
| Assessments | | | | | | | | | | | | | |
| C-SSRS Since Last | | | | X | | | | X | X | X | X | X | |
| Visitⁱ | | | | | | | | | | | | | |
| Electrocardiogram | | | | | | | | | X | X | X | | |
| Complete physical | | | | | | | | | | | X | X | |
| examination, | | | | | | | | | | | | | |
| including | | | | | | | | | | | | | |
| neurological | | | | | | | | | | | | | |
| component | | | | | | | | | | | | | |
| Abbreviated physical | | | | X | | | | X | X | X | | | |
| examination^{j, k} | | | | | | | | | | | | | |
| Vital sign | | | | X | | | | X | X | X | X | X | |
| measurements^{l} | | | | | | | | | | | | | |
| Adverse event | X | X | X | X | X | X | X | X | X | X | X | X | X |
| review and | | | | | | | | | | | | | |
| evaluation | | | | | | | | | | | | | |
| Assess for CD^{c} | X | X | X | X | X | X | X | X | X | X | X | | |
| Review participant | X | X | X | X | X | X | X | X | X | X | X | X | |
| safety card^{m} | | | | | | | | | | | | | |
| Safety Laboratory | | | | | | | | | | | | | |
| Testsⁿ | | | | | | | | | | | | | |
| Serum pregnancy^{o} | | | | | | | | | | | X | | X |
| Urine pregnancy^{o} | | | | X | | | | X | X | X | | X | |
| Clinical chemistry | | | | X | | | | X | X | X | X | X | |
| Hematology | | | | X | | | | X | X | X | X | | |
| Coagulation panel | | | | X | | | | X | X | X | X | X | |
| Urinalysis | | | | X | | | | X | X | X | X | X | |
| PK/PD Tests^{p} | | | | | | | | | | | | | |
| PK | | | | X^{q} | | | | X^{r} | X^{q} | X^{q} | | X^{q} | |
| PD: AP activity and | | | | X | | | | X | X | X | | X | |
| Bb^{s} | | | | | | | | | | | | | |
| Biomarker Tests | | | | | | | | | | | | | |
| Anti-AChR antibody | | | | X | | | | X | X | X | X | X | |
| Factor D, C3, CP | | | | X | | | | X | X | X | X | X | |
| activity, nongenetic | | | | | | | | | | | | | |
| exploratory | | | | | | | | | | | | | |
| biomarkers (eg, | | | | | | | | | | | | | |
| MuSK, LRP4)^{t} | | | | | | | | | | | | | |
| Other | | | | | | | | | | | | | |
| Concomitant | X | X | X | X | X | X | X | X | X | X | X | X | X |
| medication | | | | | | | | | | | | | |
| Nonpharmacologic | X | X | X | X | X | X | X | X | X | X | X | X | X |
| treatments and | | | | | | | | | | | | | |
| therapies | | | | | | | | | | | | | |

^{a}The ETP begins after the Day 57 (Week 8) Visit; the first visit will occur at Day 64 (Week 9). Participants will take the study intervention at home at this time point. The next in clinic visit will be at Week 12.
^{b}If a participant is discontinued from the study during the ETP, an ED Visit will be performed, and then an EOS Visit will be performed 30 (±2) days after the last dose of study intervention.
^{c}Evaluation of CD must be performed as soon as possible, within 48 hours, of notification to the Investigator of symptom onset. If CD occurs between scheduled visits, only the assessments for the CD Visit are needed. If CD occurs on a scheduled visit, all scheduled assessments should be performed for that visit as well as for the evaluation of CD. Additional evaluation visits may be scheduled at the discretion of the Investigator.
^{d}Ensure kit and lot number are recorded on the drug accountability log.
^{e}Dosing is by mouth twice daily throughout the ETP.
^{f}MG assessments should be performed at approximately the same time of day by a properly trained Clinical Evaluator (preferably the same evaluator). The MG-ADL should always be performed first, followed by the QMG.
Note:
A Clinical Evaluator may be a neurologist, neurologist in training, or delegated member of the investigational site staff who has been certified in administering the assessments.
^{g}The QMG assessment should be performed by a properly trained Clinical Evaluator (preferably the same evaluator) throughout the study. If a participant is taking a cholinesterase inhibitor, the dose must be withheld for at least 8-12 hours prior to the assessment and, whenever possible, the time from the last dose to the QMG assessment should be kept similar between visits.
^{h}The MG-ADL is required to be performed first, followed by the QMG. The MG-ADL assessment should be performed by a properly trained Clinical Evaluator (preferably the same evaluator) throughout the study. The recall period for MG-ADL is the preceding 7 days or since the last visit if the visit interval is less than 7 days. The MG-ADL will be assessed by phone at Weeks 9, 10, 11, 13, 14, and 15.
ⁱC-SSRS will be assessed Since Last Visit.
^{j}To be performed, if necessary, on the basis of the participant's health status and the clinical judgment of the Investigator.
^{k}A symptom based neurologic examination should be performed if the participant has any complaints or clinical findings attributable to the central nervous system and if positive for findings, a full neurologic examination will need to be performed at that assessment time point and at future time points as needed (determined by the Investigator).
^{l}Vital sign measurements will include systolic and diastolic blood pressure (millimeters of mercury [mmHg]), heart rate (beats/minute), and temperature (degrees Celsius [° C.] or degrees Fahrenheit [° F.]). On dosing days, vital signs will be taken before study intervention administration and after the participant has been resting for at least 5 minutes.
^{m}Participants will be given a safety card prior to the first dose of study intervention. At each visit throughout the study, investigational site staff will ensure that the participant has the safety card and will review the guidelines with the participant.
ⁿSafety laboratory samples will be analyzed by the central laboratory. A list of parameters that will be obtained during the study are provided in Table 10.
^{o}Pregnancy tests must be performed on all participants of childbearing potential at the specified time points. A serum pregnancy test will be performed at the ED and EOS visits; urine pregnancy tests will be performed locally at all other specified time points. Additional pregnancy tests (urine or serum) may also be performed at any in-clinic visit at the Investigator's discretion.
^{p}Trough blood samples for serum PK will be collected predose (within 30 minutes prior to the administration of study intervention). Peak blood samples for serum PK/PD are to be taken 2 and 4 hours postdose. All collection times will be recorded in the participant's electronic case report form.
^{q}Trough (within 30 minutes predose).
^{r}Trough (within 30 minutes predose) and peak (2 hours and 4 hours postdose).
^{s}AP activity within 30 minutes predose at Weeks 12, 26, 34, and CD, and within 30 minutes predose and 2 and 4 hours postdose at Week 16. Bb sampling within 30 minutes predose at all visits.
^{t}Collect predose.
Abbreviations: AChR = acetylcholine receptor; AP = alternative pathway; Bb = Bb fragment of complement factor B; bid = twice daily; C3 = complement component 3; CD = Clinical Deterioration; CP = classical pathway; C-SSRS = Columbia Suicide Severity Rating Scale; D = Day; ED = early discontinuation; EOS = end of study; ETP = Extended Treatment Period; IL = interleukin; LRP4 = low density lipoprotein receptor-related protein 4; MG = myasthenia gravis; MG-ADL = Myasthenia Gravis Activities of Daily Living profile; MGFA-PIS = Myasthenia Gravis Foundation of America Post-Intervention Status; MMP = matrix metalloproteinase; MuSK = muscle-specific tyrosine kinase; NA = not applicable; Neuro-QoL ™ Fatigue = Neurological Quality of Life Fatigue questionnaire; PD = pharmacodynamic; PK = pharmacokinetic; QMG = Quantitative Myasthenia Gravis score for disease severity; W = Week

TABLE 9
Schedule of Activities (Open-Label Extension Period)

| | Period | | | | | | | | |
|---|---|---|---|---|---|---|---|---|---|
| | Open-label EXtension Period^{a} | | | | | | | | |
| | Study Year | | | | | | | | |
| | Year 1 | | | | | | | | |
| | (cont'd) | Year 2+ | | | | | ED^{b} | CD^{c} | EOS^{b} |
| | Study Day (D) | | | | | | | | |
| | D 365 | D 456 | D 547 | D 638 | D 729 | D 820 | NA | NA | NA |
| | Study Week (W) | | | | | | | | |
| | W 52 | W 65 | W 78 | W 91 | W 104 | W 117 | NA | NA | NA |
| | Study Window (days) | | | | | | | | |
| | ±7 | ±7 | ±7 | ±7 | ±7 | ±7 | NA | NA | ±2 |
| Administration of Study Intervention | | | | | | | | | |
| Dispensation of study intervention^{d} | X | X | X | X | X | X | | X | |
| Administration of ALXN2050^{e} | X | X | X | X | X | X | | X | |
| Efficacy Assessments | | | | | | | | | |
| QMG^{f, g} | X | X | X | X | X | X | X | X | |
| MG-ADL^{f, h} | X | X | X | X | X | X | X | X | |
| MGFA-PIS | X | | X | | X | X | X | X | |
| Neuro-QOL ™ Fatigue | X | X | X | X | X | X | X | X | |
| Safety Assessments | | | | | | | | | |
| C-SSRS Since Last Visitⁱ | X | X | X | X | X | X | X | X | |
| Electrocardiogram | | | | | | X | X | | |
| Complete physical examination, including | | | | | | X | X | X | |
| neurological component | | | | | | | | | |
| Abbreviated physical examination^{j, k} | X | X | X | X | X | | | | |
| Vital sign measurements^{l} | X | X | X | X | X | X | X | X | |
| Adverse event review and evaluation | X | X | X | X | X | X | X | X | X |
| Assess for CD^{c} | X | X | X | X | X | X | X | | |
| Review participant safety card^{m} | X | X | X | X | X | X | X | X | |
| Safety Laboratory Testsⁿ | | | | | | | | | |
| Serum pregnancy^{o} | | | | | | | X | | X |
| Urine pregnancy^{o} | X | X | X | X | X | X | | X | |
| Clinical chemistry | X | X | X | X | X | X | X | X | |
| Hematology | X | X | X | X | X | X | X | X | |
| Coagulation panel | X | X | X | X | X | X | X | X | |
| Urinalysis | X | X | X | X | X | X | X | X | |
| PK/PD Tests^{p} | | | | | | | | | |
| PK^{q} | | | | | | | | X | |
| PD: AP activity and Bb^{r} | | | | | | | | X | |
| Biomarker Tests | | | | | | | | | |
| Anti-AChR antibody | X | | | | | X | X | X | |
| Factor D, C3, CP activity, nongenetic | | | | | | | | X | |
| exploratory biomarkers (eg, MuSK, LRP4)^{r} | | | | | | | | | |
| Other | | | | | | | | | |
| Concomitant medication | X | X | X | X | X | X | X | X | X |
| Nonpharmacologic treatments and therapies | X | X | X | X | X | X | X | X | X |

^{a}The OLE Period begins after the Day 239 (Week 34) visit. The next in-clinic visit will be at Week 52.
^{b}If a participant is discontinued from the study during the OLE Period, an ED Visit will be performed, and then an EOS Visit will be performed 30 (±2) days after the last dose of study intervention.
^{c}Evaluation of CD must be performed as soon as possible, within 48 hours, of notification to the Investigator of symptom onset. If CD occurs between scheduled visits, only the assessments for the CD Visit are needed. If CD occurs on a scheduled visit, all scheduled assessments should be performed for that visit as well as for the evaluation of CD. Additional evaluation visits may be scheduled at the discretion of the Investigator.
^{d}Ensure kit and lot number are recorded on the drug accountability log.
^{e}Dosing is by mouth twice daily throughout the OLE Period.
^{f}MG assessments should be performed at approximately the same time of day by a properly trained Clinical Evaluator (preferably the same evaluator). The MG-ADL should always be performed first, followed by the QMG.
^{g}The QMG assessment should be performed by a properly trained Clinical Evaluator (preferably the same evaluator) throughout the study. If a participant is taking a cholinesterase inhibitor, the dose must be withheld for at least 8-12 hours prior to the assessment and, whenever possible, the time from the last dose to the QMG assessment should be kept similar between visits.
^{h}The MG-ADL is required to be performed first, followed by the QMG. The MG-ADL assessment should be performed by a properly trained Clinical Evaluator (preferably the same evaluator) throughout the study. The recall period for MG-ADL is the preceding 7 days or since the last visit if the visit interval is less than 7 days.
ⁱC-SSRS will be assessed Since Last Visit.
^{j}The abbreviated physical examination will be performed, if necessary, on the basis of the participant's health status and the clinical judgment of the Investigator.
^{k}A symptom based neurologic examination should be performed if the participant has any complaints or clinical findings attributable to the central nervous system and if positive for findings, a full neurologic examination will need to be performed at that assessment time point and at future time points as needed (determined by the Investigator).
^{l}Vital sign measurements will include systolic and diastolic blood pressure (millimeters of mercury [mmHg]), heart rate (beats/minute), and temperature (degrees Celsius [° C.] or degrees Fahrenheit [F]). On dosing days, vital signs will be taken before study intervention administration and after the participant has been resting for at least 5 minutes.
^{m}Participants will be given a safety card prior to the first dose of study intervention. At each visit throughout the study, investigational site staff will ensure that the participant has the safety card and will review the guidelines with the participant.
ⁿLaboratory samples will be analyzed by the central laboratory. A list of parameters that will be obtained during the study is provided in Table 10.
Note:
A Clinical Evaluator may be a neurologist, neurologist in training, or delegated member of the investigational site staff who has been certified in administering the assessments.
^{o}Pregnancy tests must be performed on all participants of childbearing potential at the specified time points. Serum pregnancy test will be performed at the ED and EOS visits; urine pregnancy tests will be performed locally at all other specified time points. Additional pregnancy tests (urine or serum) may also be performed at any visit at the Investigator's discretion.
^{p}Trough blood samples for serum PK will be collected predose (within 30 minutes prior to the administration of study intervention). All collection times will be recorded in the participant's electronic case report form.
^{q}Trough (within 30 minutes predose).
^{r}Collect predose.
Abbreviations: AChR = acetylcholine receptor; bid = twice daily; AP = alternative pathway; Bb = fragment of complement factor B; C3 = complement component 3; CD = Clinical Deterioration; CP = classical pathway; C-SSRS = Columbia Suicide Severity Rating Scale; D = Day; ED = early discontinuation; EOS = end of study; IL = interleukin; LRP4 = low density lipoprotein receptor-related protein 4; MG = myasthenia gravis; MG-ADL = Myasthenia Gravis Activities of Daily Living profile; MGFA-PIS = Myasthenia Gravis Foundation of America Post-Intervention Status; MMP = matrix metalloproteinase; MuSK = muscle-specific tyrosine kinase; NA = not applicable; Neuro-QOL ™ Fatigue = Neurological Quality of Life Fatigue questionnaire; OLE = Open-label Extension; PD = pharmacodynamic; PK = pharmacokinetic; QMG = Quantitative Myasthenia Gravis score for disease severity; W = Week

TABLE 10
Protocol-Required Laboratory Assessments
Laboratory

| Assessments | Parameters | | |
|---|---|---|---|
| Hematology | Platelet count | RBC indices: | WBC count with |
| | RBC count | Distribution width | differential: |
| | Hemoglobin | Mean corpuscular | Neutrophils |
| | Hematocrit | volume | Lymphocytes |
| | | Mean corpuscular | Monocytes |
| | | hemoglobin | Eosinophils |
| | | % Reticulocytes | Basophils |
| Clinical | BUN | AST/SGOT | Lipids: |
| Chemistry | C-reactive protein | ALT/SGPT | Total cholesterol |
| | Creatinine | Alkaline phosphatase, | Triglycerides |
| | Chloride | Gamma | LDL-c |
| | Potassium | glutamyltransferase Total | HDL-c |
| | Bicarbonate | and direct bilirubin | |
| | Sodium | Total protein | |
| | Glucose (nonfasting) | Albumin | |
| | Creatine kinase | Uric acid | |

| | |
|---|---|
| Coagulation | international normalized ratio, partial thromboplastin time, prothrombin time |
| Urinalysis | Appearance, color, specific gravity, pH, glucose, protein, leukocyte esterase, |
| | blood, ketones, bilirubin, urobilinogen, nitrite, microscopic examination (if blood |
| | or protein is abnormal) |
| Screening tests | Serum/urine beta-hCG pregnancy test (as needed for female participants of |
| | child-bearing potential) |
| | Serum follicle-stimulating hormone test (as needed for female participants who |
| | consider themselves postmenopausal) |
| | HIV-1 and HIV-2 antibodies |
| | Hepatitis B surface antigen |
| | Hepatitis C antibody |
| Complement | Pharmacodynamic assays (AP activity and Bb concentration) |
| activity | Complement biomarkers (Factor D, C3, C5b-9, Properdin, and CP activity) |
| Other | Biomarker assay (anti-AChR antibody) |
| | Pharmacokinetic assay (serum ALXN2050 concentration) |
| | Nongenetic exploratory biomarkers (e.g., MuSK, LRP4, MMP-10, IL-6) |

### Justification for Dose

Clinical PK and PD data have been generated for ALXN2050 in Phase 1 single ascending and multiple ascending dose studies in healthy volunteers (see, e.g., International Patent Publication WO. In these Phase 1 healthy volunteer studies, ALXN2050 PK exposures increased dose proportionally following single doses and in a greater than dose proportional manner following multiple doses at steady state over the dose range of 40 mg BID to 200 mg BID. Corresponding PD activity as determined by AP inhibition in the AP Wieslab assay increased with increasing exposure.

In the multiple dose study, the dosage regimens of both 120 mg BID and 200 mg BID were safe and effective, showing at least a 10-fold safety margin in both maximum plasma concentration (Cₘₐₓ) and the area under the concentration time curve from time zero to 24 hours (AUC₀₋₂₄) over the exposures achieved at the no observed adverse effect level (NOAEL) from nonclinical chronic toxicology studies. In addition, both dosage regimens provided complete (>90%) and sustained inhibition of AP activity throughout the 12-hour dosing interval. Therefore, 120 mg BID is selected as the minimum therapeutic dosage.

However, large variabilities were observed in the PK and PD data and in the established PK/PD relationship. Intersubject variability in PK and the PK/PD relationship indicated that a dosage higher than 120 mg BID, such as 180 mg BID, may be required to ensure more participants reach and maintain an ALXN2050 concentration above the threshold for 90% AP inhibition.

The relationship between exposure and response (AP activity inhibition) has not been established specifically in participants in gMG. Therefore, one of the objectives of this study is to develop the exposure response relationship in this target population and evaluate the dose that can consistently achieve >90% AP inhibition at trough steady state concentrations. Inclusion of the 120 mg BID group is needed to fully characterize this exposure response relationship among participants with gMG to inform Phase 3 dose selection. The study is designed to explore the utility of both the 120 mg BID and the 180 mg BID dosage regimens to fully characterize the PK, PD, biomarker, efficacy, and safety data in participants with gMG.

### End of Study Definition

A participant is considered to have completed the study if (1) the participant has completed all periods of the study including the last visit of the OLE Period, or (2) in the event the study is stopped early, the participant has completed all applicable periods of the study, including the EOS Visit, or (3) the participant completes the study early (and completes the EOS Visit) because the study intervention is registered or approved (in accordance with country specific regulations). The EOS is defined as the date of the last visit of the last randomized participant in the study.

### Study Population

### Inclusion Criteria

A participant must meet all inclusion criteria to be eligible to participate in the study:

### Age

Participant must be at least 18 years of age at the time of signing the informed consent form (ICF).

### Type of Participant and Disease Characteristics

1. Diagnosed with MG at least 3 months (90 days) prior to the date of the Screening Visit. Confirmation of MG must be made via the following:
   Positive serologic test for anti-AChR antibodies at the Screening Visit, and
   Abnormal neuromuscular transmission demonstrated by single fiber electromyography or repetitive nerve stimulation, or
   Positive response to an AChEI test (e.g., edrophonium chloride test), or
   Improvement of signs or symptoms related to MG during treatment with an oral AChEI, as determined by the treating physician
2. Myasthenia Gravis Foundation of America Clinical Classification Class II to IV at the Screening Visit (Table 11).

TABLE 11
MGFA Classification System

| | |
|---|---|
| Class I | Any ocular muscle weakness; may have weakness of eye closure. All other muscle strength is |
| | normal. |
| Class II | Mild weakness affecting other than ocular muscles; may also have ocular muscle weakness of |
| | any severity. |
| Class IIa | Predominantly affecting limb, axial muscles, or both. May also have lesser involvement of |
| | oropharyngeal muscles. |
| Class IIb | Predominantly affecting oropharyngeal, respiratory muscles, or both. May also have lesser or |
| | equal involvement of limb, axial muscles, or both. |
| Class III | Moderate weakness affecting other than ocular muscle; may also have ocular muscle weakness |
| | of any severity. |
| Class IIIa | Predominantly affecting limb, axial muscles, or both. May also have lesser involvement of |
| | oropharyngeal muscles. |
| Class IIIb | Predominantly affecting oropharyngeal, respiratory muscles, or both. May also have lesser or |
| | equal involvement of limb, axial muscles, or both. |
| Class IV | Severe weakness affecting other than ocular muscles; may also have ocular muscle weakness of |
| | any severity. |
| Class IVa | Predominantly affecting limb and/or axial muscles. May also have lesser involvement of |
| | oropharyngeal muscles. |
| Class IVb | Predominantly affecting oropharyngeal, respiratory muscles, or both. May also have lesser or |
| | equal involvement of limb, axial muscles, or both. |
| Class V | Defined by intubation, with or without mechanical ventilation, except when employed during |
| | routine postoperative management. Use of a feeding tube without intubation places the patient in |
| | Class IVb. |

3. MG-ADL total score must be 5 (with at least 50% of the score attributed to non-ocular elements) at the Screening Visit and at randomization (Day 1).
   Note: Enrollment of participants with MG-ADL total score <7 will be limited to approximately 10% of the total enrollment.
4. Participants receiving the allowed treatments listed in Table 12 below must have been receiving treatment and on a stable dose for the time periods specified below prior to the date of the Screening Visit, with no changes to the regimen expected during screening, the PEP, and/or the ETP.

TABLE 12
Allowed Medications and Therapies

| | Dosage Regimen Required Prior to the |
|---|---|
| Medication Name/Drug Class Name | Screening Visit |
| azathioprine (AZA) | ≥6 months (180 days) on the medication, |
| | with a stable dose for ≥2 months (60 days) |
| Other immunosuppressive therapies, such as | ≥3 months (90 days) on the medication, |
| mycophenolate mofetil (MMF), methotrexate | with a stable dose for ≥1 month (30 days) |
| (MTX), or cyclophosphamide (CP) | |
| Corticosteroids (oral), maximum dose 20 mg/day | stable dose for ≥4 weeks (28 days) |
| prednisone or equivalent | |
| Acetylcholinesterase inhibitors^{a} | stable dose for ≥2 weeks (14 days) |

Note:
If a participant has recently discontinued any of the above medications, a period of time equal to the stable dose requirement listed above for that medication (e.g., ≥2 months for AZA or ≥4 weeks for corticosteroids) must have passed prior to the first day of the Screening Period.
^{a}As needed or intermittent acetylcholinesterase inhibitor use is not permitted at any point during the study.

### Sex

5. Male or female participant.
6. Female participants of childbearing potential and male participants must follow protocol-specified contraception guidance.

### Informed Consent

7. Capable of giving signed informed consent, which includes compliance with the requirements and restrictions listed in the ICF and in this protocol.

### Other Inclusions

8. Vaccinated against meningococcal infection (Neisseria meningitidis) within 3 years prior to, or at the time of, randomization (Day 1).
   Participants who initiate study intervention less than 2 weeks after receiving a meningococcal vaccine must receive appropriate prophylactic antibiotics until at least 2 weeks after the vaccination against N meningitidis.

### Exclusion Criteria

A participant will be excluded from the study if any exclusion criteria are satisfied.

### Medical Conditions

1. Any medical condition (e.g., cardiac, pulmonary, renal, oncologic, or psychiatric) that, in the opinion of the Investigator or the Medical Monitor, might interfere with participation in the study, pose any added risk to the participant, or confound the assessment of the participant.
2. History of thymectomy, thymomectomy, or any other thymic surgery within 12 months prior to the Screening Visit.
3. Any untreated thymic malignancy, carcinoma, or thymoma.
   Participants with a history of treated thymic malignancy or carcinoma are eligible for enrollment if they meet the following conditions:
      Treatment completed >5 years prior to the Screening Visit
      No known recurrence within the 5 years prior to the Screening Visit
      No radiological indication of recurrence in a computed tomography (CT) or magnetic resonance imaging (MRI) scan, including administration of IV contrast, performed within 6 months of randomization (Day 1)
   Participants with a history of treated benign thymoma are eligible if they meet the following conditions:
      Histopathological or equivalent records indicating the diagnosis of benign thymoma
      Treatment completed >12 months prior to the Screening Visit
      No known recurrence within the 12 months prior to the Screening Visit
      No radiological indication of recurrence in a CT or MRI scan, including administration of IV contrast, performed within 6 months of randomization (Day 1)
      If adequate records confirming the diagnosis of benign thymoma are not available, the participant must satisfy the eligibility criteria for thymic malignancy or carcinoma stated above.
4. Clinical features that, in the opinion of the Investigator, are consistent with Clinical Deterioration at the time of the Screening Visit or at any time during the Screening Period prior to randomization (Day 1).
5. History of seizure.
6. History of N meningitidis infection.
7. Evidence of human immunodeficiency virus (HIV antibody positive) infection at the Screening Visit.
8. History of hypersensitivity to any ingredient contained in the study intervention.
9. Known or suspected history of drug or alcohol abuse or dependence within 1 year prior to the Screening Visit.
10. Evidence of hepatitis B (positive hepatitis surface antigen [HBsAg] or positive core antibody [anti-HBc]) with negative surface antibody [anti-HBs] or hepatitis C viral infection (HCV antibody positive, except for patients with documented successful treatment and documented sustained virologic response [SVR]) at Screening.
11. History of malignancy within 5 years of the Screening Visit, with the exception of nonmelanoma skin cancer or carcinoma in situ of the cervix that has been treated with no evidence of recurrence.
12. History of persistent or recurrent infections prior to the Screening Visit.
13. Active systemic bacterial, viral, or fungal infection within 14 days prior to study intervention administration on Day 1.
14. Presence of fever as documented by a temperature >38° C. (100.4° F.) within 7 days prior to administration of study intervention on Day 1.
15. History or presence of any risk factors for Torsades de Pointes (e.g., heart failure/cardiomyopathy or family history of Long QT Syndrome), a screening QT interval corrected using Fridericia's formula (QTcF) >450 msec for males and >470 msec for females, or receiving medications known to significantly increase the corrected QT interval (QTc).
16. Laboratory abnormalities at the Screening Visit, including:
   Alanine aminotransferase >2×the upper limit of normal (ULN)
   Direct bilirubin >2×ULN

### Prior/Concomitant Therapy

17. Use of the following within the time periods specified below:
   IVIg or SCIg within the 4 weeks (28 days) prior to the Screening Visit
   Use of PE and PP within the 4 weeks (28 days) prior to the Screening Visit
   Use of rituximab within the 6 months (180 days) prior to the Screening Visit
   Use of tacrolimus or cyclosporine within the 4 weeks (28 days) prior to the Screening Visit
18. Any previous or current treatment with complement inhibitors (eg, eculizumab, ravulizumab).
19. Use of known cytochrome P450, family 3, subfamily A (CYP3A) sensitive substrates, moderate or strong CYP3A inducers, and/or moderate or strong CYP3A inhibitors from 2 weeks or 5 half-lives, whichever is longer, prior to the first administration of study intervention on Day 1 (randomization) (Table 13).

TABLE 13
List of Prohibited Inducers, Inhibitors, and Substrates of CYP3A

| | | Table |
|---|---|---|
| Classification | Medication | Number^{a} |
| Strong CYP3A | boceprevir, cobicistat, danoprevir and ritonavir, | 3-2 |
| inhibitors | elvitegravir and ritonavir, grapefruit juice, indinavir and | |
| | ritonavir, itraconazole, ketoconazole, lopinavir and | |
| | ritonavir, paritaprevir and ritonavir and (ombitasvir and/or | |
| | dasabuvir), posaconazole, ritonavir, saquinavir and | |
| | ritonavir, telaprevir, tipranavir and ritonavir, | |
| | telithromycin, troleandomycin, voriconazole, | |
| | clarithromycin, idelalisib, nefazodone, and nelfinavir | |
| Moderate | aprepitant, ciprofloxacin, conivaptan, crizotinib, | 3-2 |
| CYP3A | cyclosporine, diltiazem, dronedarone, erythromycin, | |
| inhibitors | fluconazole, fluvoxamine, imatinib, tofisopam, and | |
| | verapamil | |
| Strong | apalutamide, carbamazepine, enzalutamide, mitotane, | 3-3 |
| inducers of | phenytoin, rifampin, and St. John's wort | |
| CYP3A | | |
| Moderate | bosentan, efavirenz, etravirine, phenobarbital, and | 3-3 |
| inducers of | primidone | |
| CYP3A | | |
| Sensitive | alfentanil, avanafil, buspirone, conivaptan, darifenacin, | 3-1 |
| substrates of | darunavir, ebastine, everolimus, ibrutinib, lomitapide, | |
| CYP3A | lovastatin, midazolam, naloxegol, nisoldipine, saquinavir, | |
| | simvastatin, sirolimus, tacrolimus, tipranavir, triazolam, | |
| | vardenafil, budesonide, dasatinib, dronedarone, | |
| | eletriptan, eplerenone, felodipine, indinavir, lurasidone, | |
| | maraviroc, quetiapine, sildenafil, ticagrelor, tolvaptan | |

^{a}Table number from the US FDA Table of Clinical CYP Inhibitors and Inducers
Note:
This list is complete as of 25 Jan. 2021. Please visit the link below for the most up-to-date information.
http://www.fda.gov/Drugs/DevelopmentApprovalProcess/DevelopmentResources/DrugInteractionsLabeling/ucm093664.htm.
Abbreviation: CYP3A = cytochrome P450, family 3, subfamily A

20. Use of selected medications known to lower the seizure threshold and/or cause seizure:
   Meperidine/pethidine
   Tramadol
   Typical (1st generation) antipsychotics
   Clozapine
   Olanzapine
   Lithium
   Tricyclic antidepressants
   Bupropion
   Aminophylline/Theophylline
21. Current treatment with a biologic medication that may affect immune system functioning, or has stopped treatment with a biologic medication that may affect immune system functioning, and 5 terminal half-lives of the biologic medication have not elapsed by the time of the Screening Visit.

### Prior/Concurrent Clinical Study Experience

22. Participation in another interventional treatment study or use of any experimental therapy within 30 days before the Screening Visit or within 5 half-lives of the study intervention, whichever is greater.

### Other Exclusions

23. Pregnant, breastfeeding, or intending to conceive during the course of the study.
24. Inability to travel to the clinic for specified visits or fulfill the logistical requirements of study intervention administration.
25. Planned surgical procedure during the course of the study.

### Lifestyle Considerations

### Medications

If a participant is taking a cholinesterase inhibitor, the dose must be withheld for at least 8-12 hours prior to the QMG assessment at time points presented in the SoA (Tables 7-9).

### Meals and Dietary Restrictions

Certain foods such as grapefruit have been shown to be inhibitors of CYP3A4 enzyme activity. Participants should refrain from consuming these foods and beverages from 2 weeks prior to the first administration of study intervention on Day 1 (randomization) until 2 weeks after the final dose of study intervention.

### Screen Failures

Screen failures are defined as participants who consent to participate in the clinical study but are not subsequently randomized. A minimal set of screen failure information is required to ensure transparent reporting of screen failure participants to meet the Consolidated Standards of Reporting Trials publishing requirements and to respond to queries from regulatory authorities. Minimal information includes demography, screen failure details (e.g., failed eligibility criteria), and any AEs, including any serious adverse events (SAEs) and any related concomitant medication, occurring during the Screening Period.

Individuals who do not meet the criteria for participation in this study (screen failure) due to a reason that is expected to resolve, or has resolved, may be rescreened based on discussion and agreement between the Investigator and the Medical Monitor.

### Study Intervention

Study intervention is defined as any investigational intervention(s), marketed product(s), placebo, or medical device(s) intended to be administered to a study participant according to the study protocol.

### Study Intervention(s) Administered

ALXN2050 drug product will be provided as tablets that are manufactured using a common blend.
   The 60 mg formulation will be used in the study.
Matching placebo will be manufactured in the same fashion, without the active ingredient. ALXN2050 and placebo tablets will be identical in appearance.
The participant will be dispensed enough tablets to, at a minimum, cover bid dosing for 30 days during the PEP and for longer intervals during the ETP and OLE. Additional details will be provided in the interactive response technology (IRT) manual.
In order to maintain the study blind for participants, Investigators, and site personnel, each of the 2 daily doses will consist of 3 tablets that in combination correspond to the assigned treatment group.
   180 mg: three 60 mg tablets
   120 mg: two 60 mg tablets and one placebo tablet
   Placebo: 3 placebo tablets
During in-clinic visits, the participant will be administered the first dose of study intervention. The second dose of study intervention is to be taken at home. Whether in the clinic or at home, the timing of administration should be consistent.
In the event of a missed dose, the participant should be instructed to take the study intervention within 6 hours of the originally scheduled time. If more than 6 hours have passed, the missed dose should be skipped. In either scenario, the next dose should be taken according to the original dosing schedule.

The specific details of study intervention are presented in Table 14.

TABLE 14
Study Intervention

| Study Intervention Name | ALXN2050 | Placebo |
|---|---|---|
| Dose formulation | tablet | tablet |
| Unit dose strength(s) | 60 mg | NA |
| Dose level(s) and | 120 mg bid or 180 mg bid | bid |
| frequency | | |
| Route of administration | oral | oral |
| Use | experimental | placebo comparator |
| Sourcing | Alexion Pharmaceuticals, Inc. | Alexion |
| | | Pharmaceuticals, Inc. |

### Prior and Concomitant Therapies

Any medication (including over-the-counter or prescription medicines, vitamins, and/or herbal supplements) or vaccine that the participant is receiving at the time of enrollment or receives during the study must be recorded along with:
Reason for use
Dates of administration, including start and end dates
Dosage information including dose and frequency

Nonpharmacologic treatments and therapies that the participant receives during the clinical study must be recorded along with:
Reason for use
Dates of administration, including start and end dates
Diagnostics administered, if applicable
Whether the treatment is ongoing

The Medical Monitor should be contacted if there are any questions regarding concomitant or prior therapy.

### Allowed Medications and Therapy

Participants receiving treatment with any of the medications listed in Table 12 must have been receiving treatment and on a stable dose for the time periods specified prior to the date of the Screening Visit. Other concomitant medications used in the treatment of gMG may be considered on a case-by-case basis by the Investigator in consultation with the Medical Monitor.

### Disallowed Medications and Therapy

The following medications and therapies are prohibited during the study:
Known CYP3A sensitive substrates, moderate or strong CYP3A inducers, and/or moderate or strong CYP3A inhibitors are prohibited throughout the study, until 1 week after the final administration of study intervention (Table 13)
IVIg or SCIg as maintenance therapy (this is allowed acutely in the setting of a Clinical Deterioration)
PE/PP as maintenance therapy (this is allowed acutely in the setting of a Clinical Deterioration)
Rituximab
Tacrolimus or cyclosporine
Other complement inhibitors
Biologic medications that may affect immune system functioning
Selected medications known to lower the seizure threshold and/or cause seizure (see the full list of these medications listed above)

### Rescue Medicine

Participants who experience Clinical Deterioration (as defined herein) during the study may be administered rescue therapy (i.e., high-dose corticosteroid, PE/PP, or IVIg) at the discretion of the Investigator. Alexion or designee should be notified within 24 hours of initiation of treatment with rescue therapy. The name, date, and time of the dosage regimen will be recorded on the participant's eCRF. Participants who require rescue medication may continue in the study at the discretion of the Investigator.

### Vaccine and Antibiotic Prophylaxis

To mitigate the potential risk of meningococcal infection, all participants must be vaccinated within 3 years prior to, or at the time of, initiating the study intervention. Vaccines against serotypes A, C, Y, W135, and B, where available, are recommended to prevent common pathogenic meningococcal serotypes. Participants who initiate study intervention treatment less than 2 weeks after receiving a meningococcal vaccine must receive treatment with appropriate prophylactic antibiotics until at least 2 weeks after vaccination.

Participants must be vaccinated or revaccinated according to current national vaccination guidelines or local practice for vaccination use with complement inhibitors. Vaccination may not be sufficient to prevent meningococcal infection. All participants should be monitored for early signs of meningococcal infection, evaluated immediately if infection is suspected, and treated with appropriate antibiotics, if necessary.

Any participant without sufficient history of these vaccines may be vaccinated or provided boosters per national or local guidelines.

Participants should be vaccinated or revaccinated against other pathogens according to current national vaccination guidelines or local practice for vaccination use as part of standard of care.

### Efficacy Assessments

### MG-ADL

The MG-ADL profile is an 8-item participant-reported scale that focuses on relevant symptoms and functional performance of ADL in patients with MG. The 8 items of the MG-ADL questionnaire were derived from symptom-based components of the original 13-item QMG scale to assess disability secondary to ocular (2 items), bulbar (3 items), respiratory (1 item), and gross motor or limb (2 items) impairment related to effects of MG. In this functional status instrument, each response is graded 0 (normal) to 3 (most severe). The MG-ADL total score ranges from 0 to 24, with higher scores indicating worse function. The recall period for the MG-ADL profile is the preceding 7 days or since the last visit if the visit interval is less than 7 days. A 2-point change in the MG-ADL total score is considered clinically meaningful.

The MG-ADL assessment should be administered by a properly trained Clinical Evaluator (preferably the same evaluator) throughout the study. It is anticipated that the form should take no more than 10 minutes to complete. The MG-ADL is required to be performed first, followed by the QMG.

### QMG

The QMG Score for Disease Severity is an objective evaluation of therapy for MG and is based on quantitative testing of sentinel muscle groups. The MGFA task force has recommended that the QMG Score be used in prospective studies of therapy for MG (see, e.g., Benatar et al. Recommendations for myasthenia gravis clinical trials. Muscle Nerve. 2012; 45(6):909-917. The QMG instrument consists of 13 items: ocular (2 items), facial (1 item), bulbar (2 items), gross motor (6 items), axial (1 item), and respiratory (1 item); each graded 0 to 3, with 3 being the most severe. The QMG total score ranges from 0 to 39, with higher scores indicating more severe disease. The QMG assessment should be performed by a properly trained Clinical Evaluator (preferably the same evaluator) throughout the study.

If a participant is taking a cholinesterase inhibitor, the dose must be withheld for at least 8 to 12 hours prior to the assessment and, whenever possible, the time from the last dose to the QMG assessment should be kept similar between visits.

### MGFA-PIS

The gMG clinical state will be assessed using a modified version of the Myasthenia Gravis Foundation of America Post-Intervention Status (MGFA-PIS) (see, e.g., Jaretzki et al. Myasthenia gravis: recommendations for clinical research standards. Task Force of the Medical Scientific Advisory Board of the Myasthenia Gravis Foundation of America. Neurology. 2000; 55(1):16-23). Change in status categories of Improved, Unchanged, or Worse, as well as the minimal manifestation (MM) state will be assessed and recorded by the Investigator or the same neurologist skilled in the evaluation of participants with gMG throughout the study. The subscores of MM, i.e., MM-0, MM-1, MM-2, and MM-3, will not be used in this study.

### Neuro-QoL™ Fatigue

The Neuro-QoL™ Fatigue is a reliable and validated brief 19-item survey of fatigue, completed by the participant (see, e.g., Celia. Measuring Quality of Life in Neurological Disorders; Final Report of the Neuro-QOL Study September 2010. 2010). Higher scores indicate greater fatigue and greater impact of MG on activities.

### Other Embodiments

While the disclosure describes specific embodiments of methods, compounds, compositions, and uses, It will be understood that further modifications can be made thereto, and this application is intended to cover any variations or adaptations thereof following, in general, the principles of the disclosure including such departures from the disclosure that come within known or customary practice within the art to which the disclosure pertains and may be applied to essential features hereinbefore set forth, and follows in the scope of the claims. Other embodiments are within the claims.

1. A method of treating myasthenia gravis (MG) in a subject, comprising administering to the subject a therapeutically effective amount of Compound 1: 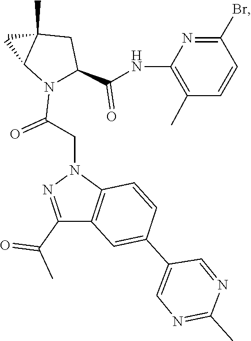 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]
or a pharmaceutically acceptable salt thereof, wherein Compound 1 or the pharmaceutically acceptable salt thereof is administered at a dose of about 60 mg to about 300 mg BID.

2. The method of claim 1, wherein the subject was diagnosed with MG at least 3 months prior to receiving treatment.

3. The method of claim 2, wherein the MG diagnosis is confirmed via a positive serologic test for anti-AChR antibodies and an abnormal neuromuscular transmission demonstrated by single fiber electromyography or repetitive nerve stimulation.

4. The method of claim 2, wherein the MG diagnosis is confirmed via a positive response to an acetylcholinesterase inhibitor (AChEI) test.

5. The method of claim 2, herein the MG diagnosis is confirmed via an improvement of signs or symptoms related to MG during treatment with an oral AChEI, as determined by a treating physician.

6. The method of any one of claims 1-5, wherein the subject is classified as having Myasthenia Gravis Foundation of America (MGFA) class II to IV disease.

7. The method of any one of claims 1-6, wherein the subject has a MG activities of daily living (MG-ADL) score of ≥5.

8. The method of any one of claims 1-7, wherein the subject has been receiving treatment with azathioprine for ≥6 months, with a stable dose of ≥2 months.

9. The method of any one of claims 1-8, wherein the subject has been receiving treatment with an immunosuppressant for ≥3 months, with a stable does for ≥1 month.

10. The method of claim 9, wherein the immunosuppressant is mycophenolate mofetil.

11. The method of claim 9, wherein the immunosuppressant is methotrexate.

12. The method of claim 9, wherein the immunosuppressant is cyclophosphamide.

13. The method of any one of claims 1-12, wherein the subject has been receiving treatment with a corticosteroid with a stable dose for ≥4 weeks.

14. The method of claim 13, wherein the corticosteroid is prednisone at a maximum dose of 20 mg/day or an equivalent thereof.

15. The method of any one of claims 1-14, wherein the subject has been receiving treatment with an AChEI with a stable dose for ≥2 weeks.

16. The method of any one of claims 1-15, wherein Compound 1 or the pharmaceutically acceptable salt thereof is orally administered.

17. The method of any one of claims 1-16, wherein Compound 1 or the pharmaceutically acceptable salt thereof is administered at a dose of about 80 mg to about 250 mg BID.

18. The method of any one of claims 1-17, wherein Compound 1 or the pharmaceutically acceptable salt thereof is administered at a dose of about 120 mg BID.

19. The method of any one of claims 1-17, wherein Compound 1 or the pharmaceutically acceptable salt thereof is administered at a dose of about 180 mg BID.

20. The method of any one of claims 1-19, wherein the treatment results in the subject experiencing a clinically meaningful improvement as determined by a reduction in Myasthenia Gravis Activities of Daily Living (MG-ADL) score.

21. The method of claim 20, wherein the MG-ADL score is reduced by at least 2 points in 4 consecutive weeks after 8 weeks of treatment, and the subject has not received a rescue therapy during treatment.

22. The method of claim 20, wherein the MG-ADL score is reduced by at least 2 points after 8 weeks of treatment.

23. The method of claim 20, wherein the MG-ADL score is reduced after 26 weeks of treatment.

24. The method of claim 23, wherein the MG-ADL score is reduced by at least 2 points after 26 weeks of treatment.

25. The method of any one of claims 1-24, wherein the treatment results in the subject experiencing a clinically meaningful improvement as determined by a reduction in quantitative Myasthenia Gravis (QMG) score after 8 weeks of treatment.

26. The method of claim 25, wherein the QMG score is reduced by at least 3 points after 8 weeks of treatment.

27. The method of claim 25, wherein the QMG score is reduced by at least 3 points in four consecutive weeks after 8 weeks of treatment, and the subject has not received a rescue therapy during treatment.

28. The method of any one of claims 1-27, wherein the treatment results in the subject experiencing a clinically meaningful improvement as determined by a reduction in Quality of Life in Neurological Disorders (Neuro-QoL™) Fatigue score after 8 weeks of treatment.

29. The method of any one of claims 1-28, wherein the treatment results in the subject experiencing a clinically meaningful improvement as determined by an improvement in the Myasthenia Gravis Foundation of America Post-Intervention Status (MGFA-PIS).

30. The method of any one of claims 1-29, wherein the MG is generalized myasthenia gravis (gMG).

31. The method of claim 30, wherein the subject is anti-AChR antibody positive.

32. The method of any one of claims 1-31, wherein the subject has a history of thymectomy, thymomectomy, or any other thymic surgery within 12 months prior to the treatment of MG.

33. The method of any one of claims 1-32, wherein the subject has an untreated thymic malignancy, carcinoma, or thymoma.

34. The method of any one of claims 1-32, wherein the subject has a history of treatment thymic malignancy or carcinoma, wherein:
(a) treatment of the thymic malignancy or carcinoma was completed more than 5 years prior to the treatment of MG;
(b) there is no known recurrence of the thymic malignancy or carcinoma within 5 years prior to the treatment of MG; and
(c) there is no radiological indication of recurrence of the thymic malignancy or carcinoma in a computed tomography (CT) or magnetic resonance imaging (MRI) scan performed within 6 months prior to the treatment of MG.

35. The method of any one of claims 1-32, wherein the subject has a history of treated benign thymoma, wherein:
(a) the subject has histopathological or equivalent records indication the diagnosis of benign thymoma;
(b) treatment of the benign thymoma was completed more than 12 months prior to the treatment of MG;
(c) there is no known recurrence of the benign thymoma within 12 years prior to the treatment of MG; and
(d) there is no radiological indication of recurrence of the benign thymoma in a CT or MRI scan performed within 6 months prior to the treatment of MG.

36. The method of any one of claims 1-32, wherein the subject does not have a history of malignancy within 5 years prior to treatment, wherein the malignancy is not nonmelanoma skin cancer or carcinoma in situ of the cervix that has been treated with no evidence of recurrence.

37. The method of any one of claims 1-36, wherein the subject is not exhibiting clinical features consistent with clinical deterioration prior to the treatment of MG.

38. The method of any one of claims 1-37, wherein the subject does not have a history of seizure.

39. The method of any one of claims 1-38, wherein the subject does not have a history of N meningitidis infection.

40. The method of any one of claims 1-39, wherein the subject is not exhibiting signs of a human immunodeficiency virus infection.

41. The method of any one of claims 1-40, wherein the subject is not exhibiting signs of a hepatitis B viral infection with negative surface antibodies.

42. The method of any one of claims 1-41, wherein the subject is not exhibiting signs of a hepatitis C viral infection; or is exhibiting signs of a hepatitis C viral infection but has been successfully treated and has a documented sustained virologic response.

43. The method of any one of claims 1-42, wherein the subject does not have a history of persistent or recurrent infections.

44. The method of any one of claims 1-43, wherein the subject did not have an active systemic bacterial, viral, or fungal infection within 14 days prior to treatment.

45. The method of any one of claims 1-44, wherein the subject does not have a history of or have risk factors for Torsades de Pointes, a QT interval corrected using Fridericia's formula (QTcF) >450 msec when the subject is male or >470 msec when the subject is female, or is receiving medication known to significantly increase the corrected QT interval (QTc).

46. The method of any one of claims 1-45, wherein the subject does not have an alanine aminotransferase level of >2×ULN.

47. The method of any one of claims 1-46, wherein the subject does not have a direct bilirubin level of >2×ULN.

48. The method of any one of claims 1-47, wherein the subject has not received intravenous immunoglobulin (IVIg) or subcutaneous immunoglobulin (SCIg) therapy within 4 weeks prior to the treatment with MG.

49. The method of any one of claims 1-48, wherein the subject has not received plasma exchange/plasmapheresis (PE/PP) treatment within 4 weeks prior to the treatment with MG.

50. The method of any one of claims 1-49, wherein the subject has not received treatment with rituximab within 6 months prior to the treatment with MG.

51. The method of any one of claims 1-50, wherein the subject has not received treatment tacrolimus or cyclosporine within 4 weeks prior to the treatment with MG.

52. The method of any one of claims 1-51, wherein the subject has not received or is not receiving treatment with a complement inhibitor.

53. The method of any one of claims 1-52, wherein the subject has not received treatment with a medication selected from a strong cytochrome P450, family 3, subfamily A (CYP3A) inhibitor; a moderate CYP3A inhibitor; a strong inducer of CYP3A; a moderate inducer of CYP3A; and a sensitive substrate of CYP3A, within the longer of two weeks or five half-lives of the medication prior to the treatment of MG.

54. The method of any one of claims 1-53, wherein the subject has not received treatment with a medication selected from meperidine, pethidine, a typical (1^{st} generation) antipsychotic, clozapine, olanzapine, lithium, a tricyclic antidepressant, bupropion, aminophylline, and theophylline.

55. The method of any one of claims 1-54, wherein the subject is not receiving treatment with a biologic medication that may affect immune system function; or the subject was previously receiving treatment with a biologic medication that may affect immune system function, and the treatment has ended for at least 5 terminal half-lives of the biologic medication.

56. The method of any one of claims 1-55, wherein the subject is restricted from consuming foods and beverages that inhibit CYP3A4 enzyme activity.

57. The method of any one of claims 1-56, wherein the subject is restricted from using a medication selected from a strong CYP3A inhibitor, a moderate CYP3A inhibitor, a strong inducer of CYP3A, a moderate inducer of CYP3A, and a sensitive substrate of CYP3A.

58. The method of any one of claims 1-57, wherein the subject is restricted from using a medication selected from meperidine, pethidine, a typical (1^{st} generation) antipsychotic, clozapine, olanzapine, lithium, a tricyclic antidepressant, bupropion, aminophylline, and theophylline.

59. The method of any one of claims 1-58, wherein the subject is restricted from using a medication selected from a strong CYP3A inhibitor, a moderate CYP3A inhibitor; a strong inducer of CYP3A; a moderate inducer of CYP3A; and a sensitive substrate of CYP3A.

60. The method of any one of claims 1-59, wherein the subject is restricted from using a medication selected from meperidine, pethidine, a typical (1^{st} generation) antipsychotic, clozapine, olanzapine, lithium, a tricyclic antidepressant, bupropion, aminophylline, and theophylline.

61. The method of any one of claims 1-60, wherein the subject is restricted from using IVIg or SCIg as a maintenance therapy.

62. The method of any one of claims 1-61, wherein the subject is restricted from using PE/PP as a maintenance therapy.

63. The method of any one of claims 1-62, wherein the subject is restricted from receiving treatment with rituximab.

64. The method of any one of claims 1-63, wherein the subject is restricted from receiving treatment with tacrolimus or cyclosporine.

65. The method of any one of claims 1-64, wherein the subject is restricted from receiving treatment with a complement inhibitor other than Compound 1 or the pharmaceutically acceptable thereof.

66. The method of any one of claims 1-65, wherein the subject is restricted from receiving treatment with a biologic medication that my affect immune system function.

67. The method of any one of claims 1-66, wherein the subject has vaccinated against meningococcal infections:
(a) within 3 years and more than two weeks prior to the treating of MG; or
(b) less than two weeks prior to the treatment of MG, and the subject is treated with an appropriate prophylactic antibiotic until at least 2 weeks after vaccination.

68. Use of Compound 1: 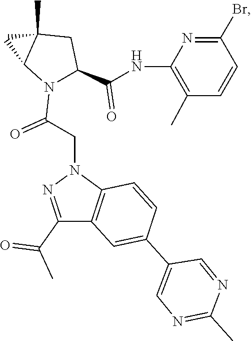 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]
or a pharmaceutically acceptable salt thereof in the manufacture of a medicament for use in a method of treating MG in a subject, wherein Compound 1 or the pharmaceutically acceptable salt thereof is formulated as a BID dosage form comprising about 60 mg to about 300 mg of Compound 1 or the pharmaceutically acceptable salt thereof.

69. The use of claim 68, wherein the subject was diagnosed with MG at least 3 months prior to receiving treatment.

70. The use of claim 69, wherein the MG diagnosis was confirmed via a positive serologic test for anti-AChR antibodies and an abnormal neuromuscular transmission demonstrated by single fiber electromyography or repetitive nerve stimulation.

71. The use of claim 69, wherein the MG diagnosis was confirmed via a positive response to an AChEI test.

72. The use of claim 69, herein the MG diagnosis was confirmed via an improvement of signs or symptoms related to MG during treatment with an oral AChEI, as determined by a treating physician.

73. The use of any one of claims 68-72, wherein the subject is classified as having MGFA class II to IV disease.

74. The use of any one of claims 68-73, wherein the subject has a MG-ADL score of ≥5.

75. The use of any one of claims 68-74, wherein the subject has been receiving treatment with azathioprine for ≥6 months, with a stable dose of ≥2 months.

76. The use of any one of claims 68-75, wherein the subject has been receiving treatment with an immunosuppressant for ≥3 months, with a stable does for ≥1 month.

77. The use of claim 76, wherein the immunosuppressant is mycophenolate mofetil.

78. The use of claim 76, wherein the immunosuppressant is methotrexate.

79. The use of claim 76, wherein the immunosuppressant is cyclophosphamide.

80. The use of any one of claims 68-79, wherein the subject has been receiving treatment with a corticosteroid with a stable dose for ≥4 weeks.

81. The use of claim 80, wherein the corticosteroid is prednisone at a maximum dose of 20 mg/day or an equivalent thereof.

82. The use of any one of claims 68-81, wherein the subject has been receiving treatment with an AChEI with a stable dose for ≥2 weeks.

83. The use of any one of claims 68-82, wherein Compound 1 or the pharmaceutically acceptable salt thereof is formulated for oral administration.

84. The use of any one of claims 68-83, wherein Compound 1 or the pharmaceutically acceptable salt thereof is formulated as a BID dosage form comprising about 80 mg to about 250 mg of Compound 1 or the pharmaceutically acceptable salt thereof.

85. The use of any one of claims 68-84, wherein Compound 1 or the pharmaceutically acceptable salt thereof is formulated as a BID dosage form comprising about 120 mg of Compound 1 or the pharmaceutically acceptable salt thereof.

86. The use of any one of claims 68-84, wherein Compound 1 or the pharmaceutically acceptable salt thereof is formulated as a BID dosage form comprising about 180 mg of Compound 1 or the pharmaceutically acceptable salt thereof.

87. The use of any one of claims 68-86, wherein the treatment results in the subject experiencing a clinically meaningful improvement as determined by a reduction in MG-ADL score.

88. The use of claim 78, wherein the MG-ADL score is reduced by at least 2 points in 4 consecutive weeks after 8 weeks of treatment, and the subject has not received a rescue therapy during treatment.

89. The use of claim 87, wherein the MG-ADL score is reduced by at least 2 points after 8 weeks of treatment.

90. The use of claim 87, wherein the MG-ADL score is reduced after 26 weeks of treatment.

91. The use of claim 90, wherein the MG-ADL score is reduced by at least 2 points after 26 weeks of treatment.

92. The use of any one of claims 68-91, wherein the treatment results in the subject experiencing a clinically meaningful improvement as determined by a reduction in QMG score after 8 weeks of treatment.

93. The use of claim 92, wherein the QMG score is reduced by at least 3 points after 8 weeks of treatment.

94. The use of claim 92, wherein the QMG score is reduced by at least 3 points in four consecutive weeks after 8 weeks of treatment, and the subject has not received a rescue therapy during treatment.

95. The use of any one of claims 68-94 wherein the treatment results in the subject experiencing a clinically meaningful improvement as determined by a reduction in Neuro-QoL Fatigue score after 8 weeks of treatment.

96. The use of any one of claims 68-95, wherein the treatment results in the subject experiencing a clinically meaningful improvement as determined by an improvement in MGFA-PIS.

97. The use of any one of claims 68-96, wherein the MG is gMG.

98. The use of claim 97, wherein the subject is anti-AChR antibody positive.

99. The use of any one of claims 68-98, wherein the subject has a history of thymectomy, thymomectomy, or any other thymic surgery within 12 months prior to the treatment of MG.

100. The use of any one of claims 68-99, wherein the subject has an untreated thymic malignancy, carcinoma, or thymoma.

101. The use of any one of claims 68-99, wherein the subject has a history of treatment thymic malignancy or carcinoma, wherein:
(a) treatment of the thymic malignancy or carcinoma was completed more than 5 years prior to the treatment of MG;
(b) there is no known recurrence of the thymic malignancy or carcinoma within 5 years prior to the treatment of MG; and
(c) there is no radiological indication of recurrence of the thymic malignancy or carcinoma in a CT or MRI scan performed within 6 months prior to the treatment of MG.

102. The use of any one of claims 68-99, wherein the subject has a history of treated benign thymoma, wherein:
(a) the subject has histopathological or equivalent records indication the diagnosis of benign thymoma;
(b) treatment of the benign thymoma was completed more than 12 months prior to the treatment of MG;
(c) there is no known recurrence of the benign thymoma within 12 years prior to the treatment of MG; and
(d) there is no radiological indication of recurrence of the benign thymoma in a CT or MRI scan performed within 6 months prior to the treatment of MG.

103. The use of any one of claims 68-99, wherein the subject does not have a history of malignancy within 5 years prior to treatment, wherein the malignancy is not nonmelanoma skin cancer or carcinoma in situ of the cervix that has been treated with no evidence of recurrence.

104. The use of any one of claims 68-103, wherein the subject is not exhibiting clinical features consistent with clinical deterioration prior to the treatment of MG.

105. The use of any one of claims 68-104, wherein the subject does not have a history of seizure.

106. The use of any one of claims 68-105, wherein the subject does not have a history of N meningitidis infection.

107. The use of any one of claims 68-106, wherein the subject is not exhibiting signs of a human immunodeficiency virus infection.

108. The use of any one of claims 68-107, wherein the subject is not exhibiting signs of a hepatitis B viral infection with negative surface antibodies.

109. The use of any one of claims 68-108, wherein the subject is not exhibiting signs of a hepatitis C viral infection; or is exhibiting signs of a hepatitis C viral infection but has been successfully treated and has a documented sustained virologic response.

110. The use of any one of claims 68-109, wherein the subject does not have a history of persistent or recurrent infections.

111. The use of any one of claims 68-110, wherein the subject did not have an active systemic bacterial, viral, or fungal infection within 14 days prior to treatment.

112. The use of any one of claims 68-111, wherein the subject does not have a history of or have risk factors for Torsades de Pointes, a QTcF >450 msec when the subject is male or >470 msec when the subject is female, or is receiving medication known to significantly increase the QTc.

113. The use of any one of claims 68-112, wherein the subject does not have an alanine aminotransferase level of >2×ULN.

114. The use of any one of claims 68-113, wherein the subject does not have a direct bilirubin level of >2×ULN.

115. The use of any one of claims 68-114, wherein the subject has not received IVIg or SCIg therapy within 4 weeks prior to the treatment with MG.

116. The use of any one of claims 68-115, wherein the subject has not received plasma PE/PP treatment within 4 weeks prior to the treatment with MG.

117. The use of any one of claims 68-116, wherein the subject has not received treatment with rituximab within 6 months prior to the treatment with MG.

118. The use of any one of claims 68-117, wherein the subject has not received treatment tacrolimus or cyclosporine within 4 weeks prior to the treatment with MG.

119. The use of any one of claims 68-118, wherein the subject has not received or is not receiving treatment with a complement inhibitor.

120. The use of any one of claims 68-119, wherein the subject has not received treatment with a medication selected from a strong CYP3A inhibitor; a moderate CYP3A inhibitor; a strong inducer of CYP3A; a moderate inducer of CYP3A; and a sensitive substrate of CYP3A, within the longer of two weeks or five half-lives of the medication prior to the treatment of MG.

121. The use of any one of claims 68-120, wherein the subject has not received treatment with a medication selected from meperidine, pethidine, a typical (1^{st} generation) antipsychotic, clozapine, olanzapine, lithium, a tricyclic antidepressant, bupropion, aminophylline, and theophylline.

122. The use of any one of claims 68-121, wherein the subject is not receiving treatment with a biologic medication that may affect immune system function; or the subject was previously receiving treatment with a biologic medication that may affect immune system function, and the treatment has ended for at least 5 terminal half-lives of the biologic medication.

123. The use of any one of claims 68-122, wherein the subject is restricted from consuming foods and beverages that inhibit CYP3A4 enzyme activity.

124. The use of any one of claims 68-123, wherein the subject is restricted from using a medication selected from a strong CYP3A inhibitor, a moderate CYP3A inhibitor, a strong inducer of CYP3A, a moderate inducer of CYP3A, and a sensitive substrate of CYP3A.

125. The use of any one of claims 68-124, wherein the subject is restricted from using a medication selected from meperidine, pethidine, a typical (1^{st} generation) antipsychotic, clozapine, olanzapine, lithium, a tricyclic antidepressant, bupropion, aminophylline, and theophylline.

126. The use of any one of claims 68-125, wherein the subject is restricted from using a medication selected from a strong CYP3A inhibitor, a moderate CYP3A inhibitor; a strong inducer of CYP3A; a moderate inducer of CYP3A; and a sensitive substrate of CYP3A.

127. The use of any one of claims 68-126, wherein the subject is restricted from using a medication selected from meperidine, pethidine, a typical (1^{st} generation) antipsychotic, clozapine, olanzapine, lithium, a tricyclic antidepressant, bupropion, aminophylline, and theophylline.

128. The use of any one of claims 68-127, wherein the subject is restricted from using IVIg or SCIg as a maintenance therapy.

129. The use of any one of claims 68-128, wherein the subject is restricted from using PE/PP as a maintenance therapy.

130. The use of any one of claims 68-129, wherein the subject is restricted from receiving treatment with rituximab.

131. The use of any one of claims 68-130, wherein the subject is restricted from receiving treatment with tacrolimus or cyclosporine.

132. The use of any one of claims 68-131, wherein the subject is restricted from receiving treatment with a complement inhibitor other than Compound 1 or the pharmaceutically acceptable thereof.

133. The use of any one of claims 68-132, wherein the subject is restricted from receiving treatment with a biologic medication that my affect immune system function.

134. The use of any one of claims 68-133, wherein the subject has vaccinated against meningococcal infections:
(a) within 3 years and more than two weeks prior to the treating of MG; or
(b) less than two weeks prior to the treatment of MG, and the subject is treated with an appropriate prophylactic antibiotic until at least 2 weeks after vaccination.

135. A compound for use in a method of treating of MG in a subject, wherein the compound is Compound 1: 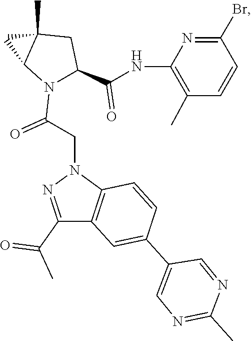 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]
or a pharmaceutically acceptable salt thereof and is formulated as a BID dosage form comprising about 60 mg to about 300 mg of the compound.

136. The compound of claim 135, wherein the subject was diagnosed with MG at least 3 months prior to receiving treatment.

137. The compound of claim 136, wherein the MG diagnosis was confirmed via a positive serologic test for anti-AChR antibodies and an abnormal neuromuscular transmission demonstrated by single fiber electromyography or repetitive nerve stimulation.

138. The compound of claim 136, wherein the MG diagnosis was confirmed via a positive response to an AChEI test.

139. The compound of claim 136, herein the MG diagnosis was confirmed via an improvement of signs or symptoms related to MG during treatment with an oral AChEI, as determined by a treating physician.

140. The compound of any one of claims 135-139, wherein the subject is classified as having MGFA class II to IV disease.

141. The compound of any one of claims 135-140, wherein the subject has a MG-ADL score of ≥5.

142. The compound of any one of claims 135-141, wherein the subject has been receiving treatment with azathioprine for ≥6 months, with a stable dose of ≥2 months.

143. The compound of any one of claims 135-142, wherein the subject has been receiving treatment with an immunosuppressant for ≥3 months, with a stable does for ≥1 month.

144. The compound of claim 143, wherein the immunosuppressant is mycophenolate mofetil.

145. The compound of claim 143, wherein the immunosuppressant is methotrexate.

146. The compound of claim 143, wherein the immunosuppressant is cyclophosphamide.

147. The compound of any one of claims 135-146, wherein the subject has been receiving treatment with a corticosteroid with a stable dose for ≥4 weeks.

148. The compound of claim 147, wherein the corticosteroid is prednisone at a maximum dose of 20 mg/day or an equivalent thereof.

149. The compound of any one of claims 135-148, wherein the subject has been receiving treatment with an AChEI with a stable dose for ≥2 weeks.

150. The compound of any one of claims 135-149, formulated for oral administration.

151. The compound of any one of claims 135-150, formulated as a BID dosage form comprising about 80 mg to about 250 mg of the compound.

152. The compound of any one of claims 135-151, formulated as a BID dosage form comprising about 120 mg of the compound.

153. The compound of any one of claims 135-151, formulated as a BID dosage form comprising about 180 mg of the compound.

154. The compound of any one of claims 135-153, wherein the treatment results in the subject experiencing a clinically meaningful improvement as determined by a reduction in MG-ADL score.

155. The compound of claim 154, wherein the MG-ADL score is reduced by at least 2 points in 4 consecutive weeks after 8 weeks of treatment, and the subject has not received a rescue therapy during treatment.

156. The compound of claim 155, wherein the MG-ADL score is reduced by at least 2 points after 8 weeks of treatment.

157. The compound of claim 156, wherein the MG-ADL score is reduced after 26 weeks of treatment.

158. The compound of claim 157, wherein the MG-ADL score is reduced by at least 2 points after 26 weeks of treatment.

159. The compound of any one of claims 135-158, wherein the treatment results in the subject experiencing a clinically meaningful improvement as determined by a reduction in QMG score after 8 weeks of treatment.

160. The compound of claim 159, wherein the QMG score is reduced by at least 3 points after 8 weeks of treatment.

161. The compound of claim 159, wherein the QMG score is reduced by at least 3 points in four consecutive weeks after 8 weeks of treatment, and the subject has not received a rescue therapy during treatment.

162. The compound of any one of claims 135-161 wherein the treatment results in the subject experiencing a clinically meaningful improvement as determined by a reduction in Neuro-QoL Fatigue score after 8 weeks of treatment.

163. The compound of any one of claims 135-162, wherein the treatment results in the subject experiencing a clinically meaningful improvement as determined by an improvement in MGFA-PIS.

164. The compound of any one of claims 135-163, wherein the MG is gMG.

165. The compound of claim 164, wherein the subject is anti-AChR antibody positive.

166. The compound of any one of claims 135-165, wherein the subject has a history of thymectomy, thymomectomy, or any other thymic surgery within 12 months prior to the treatment of MG.

167. The compound of any one of claims 135-166, wherein the subject has an untreated thymic malignancy, carcinoma, or thymoma.

168. The compound of any one of claims 135-166, wherein the subject has a history of treatment thymic malignancy or carcinoma, wherein:
(a) treatment of the thymic malignancy or carcinoma was completed more than 5 years prior to the treatment of MG;
(b) there is no known recurrence of the thymic malignancy or carcinoma within 5 years prior to the treatment of MG; and
(c) there is no radiological indication of recurrence of the thymic malignancy or carcinoma in a CT or MRI scan performed within 6 months prior to the treatment of MG.

169. The compound of any one of claims 135-166, wherein the subject has a history of treated benign thymoma, wherein:
(a) the subject has histopathological or equivalent records indication the diagnosis of benign thymoma;
(b) treatment of the benign thymoma was completed more than 12 months prior to the treatment of MG;
(c) there is no known recurrence of the benign thymoma within 12 years prior to the treatment of MG; and
(d) there is no radiological indication of recurrence of the benign thymoma in a CT or MRI scan performed within 6 months prior to the treatment of MG.

170. The compound of any one of claims 135-166, wherein the subject does not have a history of malignancy within 5 years prior to treatment, wherein the malignancy is not nonmelanoma skin cancer or carcinoma in situ of the cervix that has been treated with no evidence of recurrence.

171. The compound of any one of claims 135-170, wherein the subject is not exhibiting clinical features consistent with clinical deterioration prior to the treatment of MG.

172. The compound of any one of claims 135-171, wherein the subject does not have a history of seizure.

173. The compound of any one of claims 135-172, wherein the subject does not have a history of N meningitidis infection.

174. The compound of any one of claims 135-173, wherein the subject is not exhibiting signs of a human immunodeficiency virus infection.

175. The compound of any one of claims 135-174, wherein the subject is not exhibiting signs of a hepatitis B viral infection with negative surface antibodies.

176. The compound of any one of claims 135-175, wherein the subject is not exhibiting signs of a hepatitis C viral infection; or is exhibiting signs of a hepatitis C viral infection but has been successfully treated and has a documented sustained virologic response.

177. The compound of any one of claims 135-176, wherein the subject does not have a history of persistent or recurrent infections.

178. The compound of any one of claims 135-177, wherein the subject did not have an active systemic bacterial, viral, or fungal infection within 14 days prior to treatment.

179. The compound of any one of claims 135-178, wherein the subject does not have a history of or have risk factors for Torsades de Pointes, a QTcF >450 msec when the subject is male or >470 msec when the subject is female, or is receiving medication known to significantly increase the QTc.

180. The compound of any one of claims 135-179, wherein the subject does not have an alanine aminotransferase level of >2×ULN.

181. The compound of any one of claims 135-180, wherein the subject does not have a direct bilirubin level of >2×ULN.

182. The compound of any one of claims 135-181, wherein the subject has not received IVIg or SCIg therapy within 4 weeks prior to the treatment with MG.

183. The compound of any one of claims 135-182, wherein the subject has not received PE/PP treatment within 4 weeks prior to the treatment with MG.

184. The compound of any one of claims 135-183, wherein the subject has not received treatment with rituximab within 6 months prior to the treatment with MG.

185. The compound of any one of claims 135-184, wherein the subject has not received treatment tacrolimus or cyclosporine within 4 weeks prior to the treatment with MG.

186. The compound of any one of claims 135-185, wherein the subject has not received or is not receiving treatment with a complement inhibitor.

187. The compound of any one of claims 135-186, wherein the subject has not received treatment with a medication selected from a strong CYP3A inhibitor; a moderate CYP3A inhibitor; a strong inducer of CYP3A; a moderate inducer of CYP3A; and a sensitive substrate of CYP3A, within the longer of two weeks or five half-lives of the medication prior to the treatment of MG.

188. The compound of any one of claims 135-187, wherein the subject has not received treatment with a medication selected from meperidine, pethidine, a typical (1^{st} generation) antipsychotic, clozapine, olanzapine, lithium, a tricyclic antidepressant, bupropion, aminophylline, and theophylline.

189. The compound of any one of claims 135-188, wherein the subject is not receiving treatment with a biologic medication that may affect immune system function; or the subject was previously receiving treatment with a biologic medication that may affect immune system function, and the treatment has ended for at least 5 terminal half-lives of the biologic medication.

190. The compound of any one of claims 135-189, wherein the subject is restricted from consuming foods and beverages that inhibit CYP3A4 enzyme activity.

191. The compound of any one of claims 135-190, wherein the subject is restricted from using a medication selected from a strong CYP3A inhibitor, a moderate CYP3A inhibitor, a strong inducer of CYP3A, a moderate inducer of CYP3A, and a sensitive substrate of CYP3A.

192. The compound of any one of claims 135-191, wherein the subject is restricted from using a medication selected from meperidine, pethidine, a typical (1^{st} generation) antipsychotic, clozapine, olanzapine, lithium, a tricyclic antidepressant, bupropion, aminophylline, and theophylline.

193. The compound of any one of claims 135-192, wherein the subject is restricted from using a medication selected from a strong CYP3A inhibitor, a moderate CYP3A inhibitor; a strong inducer of CYP3A; a moderate inducer of CYP3A; and a sensitive substrate of CYP3A.

194. The compound of any one of claims 135-193, wherein the subject is restricted from using a medication selected from meperidine, pethidine, a typical (1^{st} generation) antipsychotic, clozapine, olanzapine, lithium, a tricyclic antidepressant, bupropion, aminophylline, and theophylline.

195. The compound of any one of claims 135-194, wherein the subject is restricted from using IVIg or SCIg as a maintenance therapy.

196. The compound of any one of claims 135-195, wherein the subject is restricted from using PE/PP as a maintenance therapy.

197. The compound of any one of claims 135-196, wherein the subject is restricted from receiving treatment with rituximab.

198. The compound of any one of claims 135-197, wherein the subject is restricted from receiving treatment with tacrolimus or cyclosporine.

199. The compound of any one of claims 135-198, wherein the subject is restricted from receiving treatment with a complement inhibitor other than Compound 1 or the pharmaceutically acceptable thereof.

200. The compound of any one of claims 135-199, wherein the subject is restricted from receiving treatment with a biologic medication that my affect immune system function.

201. The compound of any one of claims 135-200, wherein the subject has vaccinated against meningococcal infections:
(a) within 3 years and more than two weeks prior to the treating of MG; or
(b) less than two weeks prior to the treatment of MG, and the subject is treated with an appropriate prophylactic antibiotic until at least 2 weeks after vaccination.

202. A kit for treating MG in a subject, comprising:
(a) a dose of Compound 1: 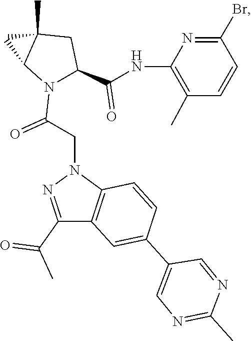 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]
or a pharmaceutically acceptable salt thereof; and
(b) instructions for using Compound 1 or the pharmaceutically acceptable salt thereof according to the method of any one of claims 1-67.
